(19) United States
(12) Patent Application Publication
(10) Pub. No.: US 2024/0108634 A1
(43) Pub. Date: Apr. 4, 2024

(54) COMPOSITIONS AND METHODS FOR INHIBITING YAP

(71) Applicant: The United States Government as represented by the Department of Veterans Affairs, Washington, DE (US)

(72) Inventors: Joseph Gera, North Hills, CA (US); Jacquelyn T. Saunders, Los Angeles, CA (US); Brent Holmes, Los Angeles, CA (US); Angelica Benavides-Serrato, Los Angeles, CA (US); Robert N. Nishimura, Los Angeles, CA (US)

(21) Appl. No.: 18/274,293

(22) PCT Filed: Jan. 26, 2022
(86) PCT No.: PCT/US2022/013786

§ 371 Date: Jul. 26, 2023

Related U.S. Application Data
(60) Provisional application No. 63/141,718, filed on Jan. 26, 2021.

(51) Int. Cl. A61K 31/55; A61K 45/06; A61P 35/00

(52) U.S. Cl. CPC A61K 31/55; A61K 45/06; A61P 35/00

(57) ABSTRACT

Described herein are compositions and their use in methods and pharmaceutical compositions for inhibiting the binding between Yes-associated protein and TEA domain. Also, described herein are methods of using said compositions capable of inhibiting the binding between Yes-associated protein and TEA domain to treat cancer, inhibit cell proliferation, cell migration, cell invasiveness and tumor growth.

### CROSS REFERENCE TO RELATED APPLICATIONS

This application claims the benefit of U.S. Provisional Application No. 63/141,718, filed Jan. 26, 2021. The content of this earlier filed application is hereby incorporated by reference herein in its entirety.

### STATEMENT REGARDING FEDERALLY FUNDED RESEARCH

This invention was made with government support under grant number CA217820 awarded by National Institutes of Health. The government has certain rights in the invention.

### BACKGROUND

Glioblastoma is an aggressive type of cancer that occurs in the brain or spinal cord. The cause of most cases of glioblastoma is unknown and there is not known method of preventing this cancer. Treatment consists of surgery after chemotherapy and radiation therapy have been used. Despite treatment, the cancer usually recurs with the duration of survival following diagnosis about 12 to 15 months. Thus, new treatment strategies are needed.

### SUMMARY

Disclosed herein are methods of treating cancer in a subject, the methods comprising: administering to the subject a therapeutically effective amount of compound capable of inhibiting the binding between Yes-associated protein (YAP) and TEA domain (TEAD), thereby reducing YAP levels.

Disclosed herein are methods of reducing Yes-associated protein (YAP) levels in a subject, the methods comprising: administering to the subject a therapeutically effective amount of compound capable of inhibiting the binding between Yes-associated protein (YAP) and TEA domain (TEAD), thereby reducing YAP levels.

Disclosed herein are methods of reducing Yes-associated protein (YAP) expression in a subject, the methods comprising: administering to the subject a therapeutically effective amount of compound capable of inhibiting the binding between Yes-associated protein (YAP) and TEA domain (TEAD), thereby reducing YAP expression.

Disclosed herein are methods of inhibiting cell proliferation, cell migration, or cell invasiveness, the methods comprising: contacting a cell or tissue or administering to a subject a therapeutically effective amount of compound capable of inhibiting the binding between Yes-associated protein (YAP) and TEA domain (TEAD), thereby reducing YAP levels.

Disclosed herein are methods of inhibiting tumor growth, the methods comprising: contacting a cell or tissue or administering to a subject a therapeutically effective amount of compound capable of inhibiting the binding between Yes-associated protein (YAP) and TEA domain (TEAD), thereby reducing YAP levels.

Disclosed herein are methods of treating glioblastoma in a subject, the methods comprising: administering to the subject a therapeutically effective amount of compound capable of inhibiting the binding between Yes-associated protein (YAP) and TEA domain (TEAD), thereby reducing YAP levels.

Disclosed herein are methods comprising: a) obtaining or having obtained a sample comprising tumor cells from a cancer patient; b) measuring the level of Yes-associated protein (YAP) in the tumor cells of the sample; c) identifying the cancer patient as a suitable candidate for treatment with a composition comprising:

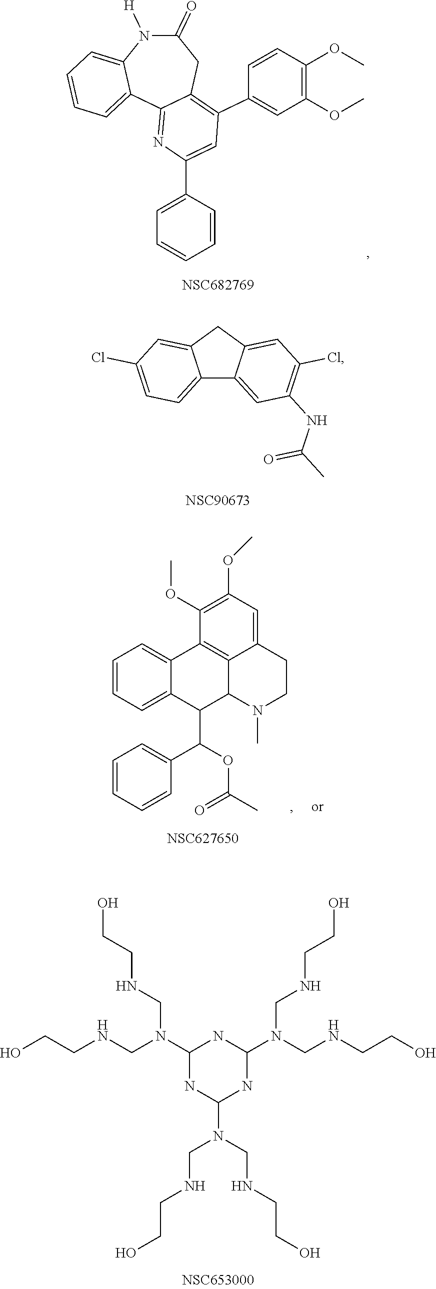 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

when the level of YAP is higher than a level of YAP in a control sample and identifying the cancer patient as an unsuitable candidate for treatment with a composition comprising:

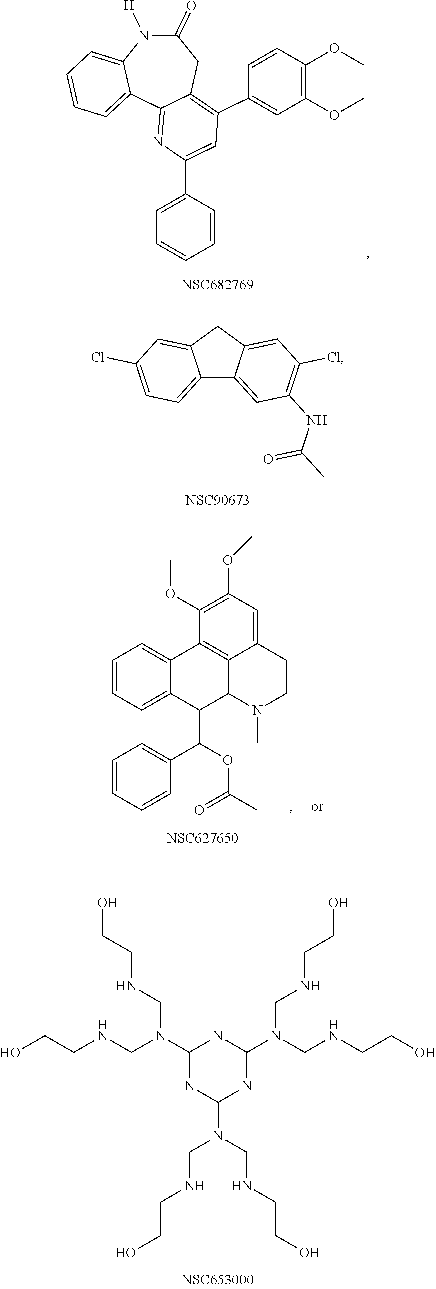 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

when the level of YAP is the same or lower than a level in a control sample; and d) administering the composition comprising:

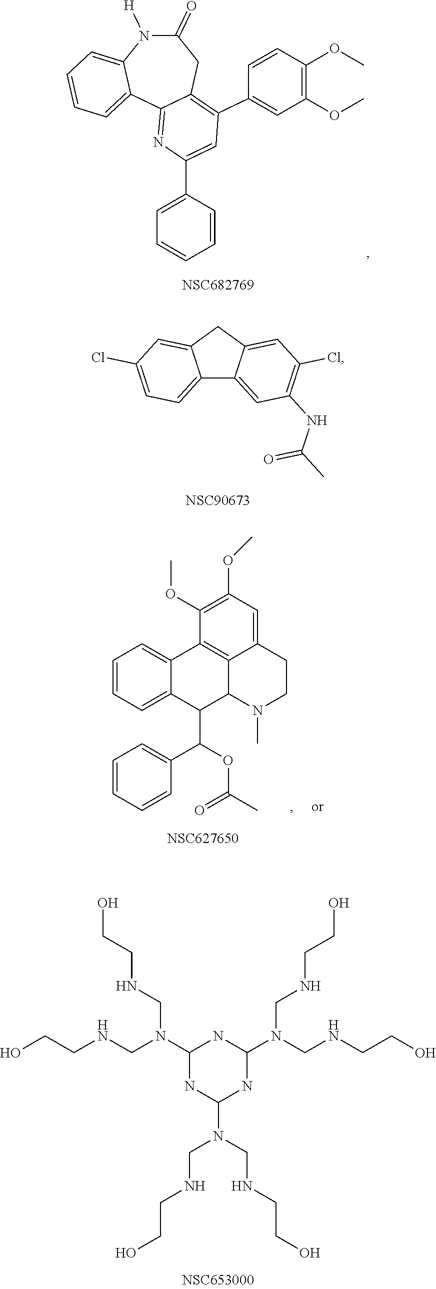 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

to the cancer patient identified as the suitable candidate, and not administering the composition comprising:

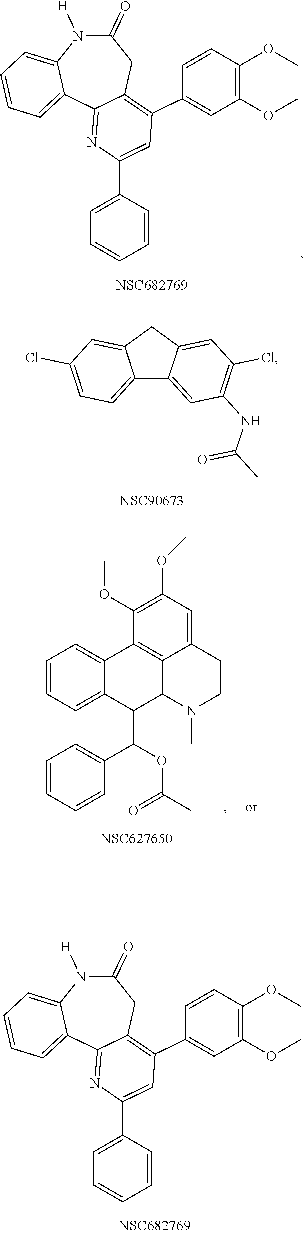 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; COc1ccc(-c2cc(-c3ccccc3)nc3c2CC(=O)Nc2ccccc2-3)cc1OC]

to the cancer patient identified as the unsuitable candidate.

Other features and advantages of the present compositions and methods are illustrated in the description below, the drawings, and the claims.

### BRIEF DESCRIPTION OF THE DRAWINGS

FIGS. 1A-G show the identification of a YAP1-TEAD1 binding interface inhibitor. FIG. 1A shows a schematic diagram of the yeast two-hybrid configuration used for screening. The Gal4 DBD was fused to residues 50-171 of human YAP1 and the Gal4 AD was fused to residues 194-411 of human TEAD1. These fusions were expressed in yeast containing reporters harboring Gal4 upstream activating sequences (UAS). FIG. 1B shows the high-throughput screening of compounds which inhibit YAP1-TEAD1 association. Yeast expressing either p53-DBD and SV40 large T antigen-AD fusions or YAP1-DBD and TEAD1-AD fusions were plated on selective media. Compounds were pinned onto the plate surfaces by robotic transfer and subsequently examined for halo formation (indicated by red circles). Compounds inhibiting YAP1-TEAD1 mediated growth on selective media while having no effect on p53-T antigen mediated yeast growth were considered specific. Compounds which blocked the growth of both strains were considered nonspecific and were classified as having general antifungal properties. FIG. 1C shows the compound structures of candidate YAP-TEAD inhibitors. FIG. 1D shows that NSC682769 blocks in vitro YAP-TEAD binding in a GST pull-down assay. His-tagged YAP (amino acids 50-171 of Accession No. NP_006097) was used in a GST pull-down assay using GST-TEAD (amino acids 194-411 of Accession No. NP_068780) immobilized on glutathione resin. Pull-down products were analyzed by SDS-PAGE and silver staining. FIG. 1E shows LN229 cells expressing HA-YAP and myc-TEAD were incubated with the indicated concentrations of inhibitors for 24 h and the presence of myc-TEAD in the HA-YAP immunoprecipitates was probed. Cells treated with antimycin A are shown as a negative control. Verteporfin (VP) at equimolar concentrations was ineffective at inhibiting the interaction and required cell treatment at 4 μM for observable blockade. FIG. 1F shows that purified recombinant HA-tagged full-length human YAP1 was added to uncross-linked control, IRES-J007 (hnRNP A1 inhibitor) and NSC682769 coupled beads and the amount of bound YAP was assessed by immunoblot analyses. FIG. 1G shows the surface plasmon resonance analyses of immobilized full-length YAP binding to the indicated concentrations of NSC682769 analyte. Raw binding sensorgrams obtained at each concentration were fitted to a 1:1 binding model and Kon, Koff and KD were calculated by simultaneous non-linear regression analyses.

FIGS. 2A-G show that NSC682769 selectively inhibits YAP-TEAD signaling in GBM. FIG. 2A shows the effects of NSC682769 following 18 h incubation at the indicated concentrations in LN229, T98G and the patient-derived GBM line GBM39. FIG. 2B shows inhibition of YAP expression in LN229 and GBM39 cells. Levels of total YAP were determined via ELISA. NSC682769 inhibited total YAP with IC50's of 11.8 and 5.1 nM, in LN229 and GBM39 cells, respectively. FIG. 2C shows LN229 cells expressing HA-tagged YAP1 that were treated with NSC682769 for 18 h as indicated and a-HA antibody used to immunoprecipitate HA-YAP1. Immunoprecipitates were immunoblotted for the indicated proteins. FIG. 2D shows inhibition of YAP-target gene (CTGF & Cyr61) expression by NSC682769 in LN229 and GBM 39 cells. Cells were treated with the indicated concentrations of inhibitor for 18 h and mRNA extracted for real time RT-PCR analyses. Measurements were performed in quadruplicate and means and+S.D. are shown. FIG. 2E shows LN229 or GBM39 expressing a multimerized TEAD binding site luciferase reporter (HOP-flash) that were treated with the indicated concentrations of NSC682769 for 18 h and YAP-TEAD transcriptional activity determined via luciferase assays. FIG. 2F shows the effects of NSC682769 on YAP cytoplasmic/nuclear accumulation in LN229 and GBM39 cells. Cells were treated with (+) or without (−) NSC682769 (100 nM, 18 h) and harvested and separated into cytoplasmic and nuclear cellular fractions. Cytoplasmic versus nuclear material was subsequently immunoblotted for the indicated proteins. FIG. 2G shows indirect immunofluorescence analyses of YAP localization following exposure of LN229 and GBM39 cells to NSC682769 (50 nM, 6 h). Values in top right corner of panels correspond to the percentage of nuclear localized YAP. Cells were grown on coverslips and treated with NSC682769. Cells were permeabilized and stained using primary antibody to YAP1 and FITC-conjugated secondary. Nuclei were stained with DAPI. Scale bar, 20 μm.

FIGS. 3A-E show that NSC682769 inhibits proliferation, anchorage-independent growth, motility, invasive potential and induced apoptosis in GBM. FIG. 3A shows inhibition of LN229 and patient-derived GBM line GBM39 proliferation following culture with NSC682769 (blue, 0.1 μM; red, 1 μM; green, 2 μM) for the indicated time points (*, P<0.05). FIG. 3B shows inhibition of anchorage-independent growth by NSC682769. Cells were layered in soft agar to evaluate anchorage-independent growth in the presence of the indicated concentrations of inhibitor and colonies counted following 14 days of growth. Representative crystal violet stained images are shown below. Data represent mean+S.D. of three independent experiments. FIG. 3C shows the migration of LN229 and GBM39 cells in the presence of NSC682769 at the concentrations shown. Cells were placed in Boyden chambers and allowed to migrate towards BSA (white bars), vitronectin (light yellow bars), or fibronectin (dark yellow bars) (*, P<0.05; n=3). FIG. 3D shows the invasive potential of LN229 or GBM39 cells in the presence of the indicated concentrations of NSC682769 migrating through matrigel. Representative crystal violet stained images are shown (200×). Data represent mean+S.D. of three independent experiments. FIG. 3E shows the percent apoptotic cells in LN229 and GBM39 cells treated with the indicated concentrations of NSC682769 at 48 h as determined via annexin V-FITC/PI staining and flow cytometry. Graphical data shown below represent mean+S.D. (late apoptosis; annexin V-positive, PI-positive) of three independent experiments.

FIGS. 4A-E show that elevated nuclear YAP levels correlate with NSC682769 sensitivity in GBM. FIG. 4A shows nuclear and cytoplasmic accumulation of YAP in the indicated GBM lines. Nuclear and cytoplasmic fractions were immunoblotted for YAP, HSP90 and lamin B2. FIG. 4B show quantification of nuclear YAP expression levels from a as determined by densitometric analyses. Relative nuclear YAP1 expression in U87 cells was arbitrarily assigned a value of 1. Nuclear YAP expression in the cell lines is shown as mean+S.D., n=3. FIG. 4C shows that relative cell proliferation was determined via Cell Titer-Glo® luminescent assays on the indicated GBM lines with increasing concentrations of NSC682769 at 72 h. FIG. 4D shows the correlation between NSC682769 IC₅₀ and relative nuclear YAP expression as determined for the GBM cell lines treated with NSC682769 for 72 h and shown as means of 3-5 individual experiments. Relative nuclear YAP expression was obtained from FIG. 4B above. FIG. 4E shows the differential sensitivities of parental T98G, shRNA YAP expressing T98G and hYAP1 overexpressing T98G cells exposed to NSC682769 at the indicated concentrations at 72 h. Data represent mean±S.D. of three independent experiments (*, P<0.05).

FIGS. 5A-G show that NSC682769 inhibits GBM tumor growth in mice. FIG. 5A shows the effects of NSC682769 on tumor growth in SCID mice implanted with subcutaneous LN229 xenografts and treated with the indicated schedules of vehicle, NSC682769 (5 mg/kg/d) and NSC682769 (20 mg/kg/d). treatment day; *, P<0.05, significantly different from vehicle (n=5 per group). FIG. 5B shows the overall survival of mice harboring subcutaneous LN229 tumors receiving the indicated treatment schedules of NSC682769. FIG. 5C shows the tumor weights of harvested tumors from xenografted mice treated with the indicated treatment schedules of NSC682769. *, P<0.05, significantly different from vehicle. FIG. 5D shows the YAP protein levels in tumors from harvested tumors. Protein levels were quantified by Western analyses (inset; representative immunoblots of YAP and actin levels from tumors from the indicated tumor treatment groups), and normalized to actin levels of harvested tumors from mice with the corresponding treatments as indicated and described in Materials and Methods. Values are means±S.D., *, P<0.05, significantly different from vehicle, NSC682769 (5 mg/kg/d) and NSC682769 (20 mg/kg/d). FIG. 5E shows inhibition of YAP-dependent gene transcription in harvested xenografts treated with the indicated regimens. CTGF and Cyr61 mRNA levels were determined via real-time RT-PCR measurements. *, P<0.05 significantly different from vehicle, NSC682769 (5 mg/kg/d) and NSC682769 (20 mg/kg/d). FIG. 5F shows that Ki-67 positive cells were identified via immunohistochemical staining of sections prepared from harvested tumors at day 12 following initiation of treatment regimens. *, P<0.05 significantly different from vehicle. Scale bar, 20 μm. FIG. 5G shows apoptotic cells were identified by TUNEL assays of sections prepared from harvested tumors at day 12 following initiation of treatment regimens. Data are expressed as the number of positive apoptotic bodies (brown, indicated by arrows) divided by high power field (hpf; 10-12 hpf/tumor). Values are means+S.D., *, P<0.05. Scale bar, 20 μm.

FIG. 6 shows TUNEL staining of human neurons treated with increasing concentrations of NSC682769 following 48 h exposures. Data shown are mean+S.D., n=3.

FIG. 7 shows peripheral blood RBC and WBC counts in mice (5 mice/group) treated with daily IP injections (5 days) of 0, 5 or 20 mg/kg/d of NSC682769. Data are expressed as percent of control mice that received vehicle and assigned 100%.

FIG. 8 shows YAP-target gene (Ctgf & Cyr61) expression in R0E3 cells derived from tumors of double transgenic GFAP-EGFRvIII; GFAP-Cre/Rictor^{loxP/loxP} mice. mRNA was extracted for real time RT-PCR analyses. Measurements were performed in quadruplicate and means and+S.D. are shown.

FIGS. 9A-B show GEMM survival and brain/blood plasma concentrations following treatment with NSC682769. FIG. 9A shows the effects of NSC682769 on Tg EGFRVIII×Rictor overexpressors upon twice weekly treatment at 5 or 20 mg/kg as indicated. wt and EGFRvIII mice had 100% tumor-free survival in these experiments. FIG. 9B shows that NSC682769 crosses the BBB. C57/BL6 mice were administered NSC682769 IV and mean brain and plasma concentrations were determined via HPLC analyses. (n=5).

### DETAILED DESCRIPTION

Many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

As used in the specification and in the claims, the term “comprising” can include the aspects “consisting of” and “consisting essentially of.” “Comprising” can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. The subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder (e.g., cancer). The term “patient” includes human and veterinary subjects. In some aspects of the disclosed methods, the “patient” has been diagnosed with a need for treatment for cancer, such as, for example, prior to the administering step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment can also be administered to a subject to ameliorate one more signs of symptoms of a disease, disorder, and/or condition. For example, the disease, disorder, and/or condition can be relating to cancer or glioblastoma.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

Glioblastoma is the most aggressive of CNS tumors with the median survival of 12-17 months, necessitating the development of novel therapeutics (Cloughesy T F, Cavenee W K, Mischel P S (2014) Glioblastoma: from molecular pathology to targeted treatment. Annu Rev Pathol 9: 1-25). Aberrant regulation of the Hippo signaling pathway has been observed in several cancers including glioblastoma (Wang Y, et al., Cancer Genome Atlas Research N, Camargo F, Liang H (2018) Comprehensive Molecular Characterization of the Hippo Signaling Pathway in Cancer. Cell Rep 25: 1304-1317; and Meng Z, et al. (2016) Genes Dev 30: 1-17). Gene regulatory network and comprehensive expression analyses of brain tumor samples found that YAP and the transcriptional coactivator with PDZ-binding motif (TAZ) were major drivers of GBM transformation (Bhat K P, et al. (2011) Genes Dev 25: 2594-2609; Orr B A, et al. (2011) J Neuropathol Exp Neurol 70: 568-577; and Liu M, et al. (2017) Lab Invest 97: 1354-1363). Additionally, clinical studies have demonstrated high expression levels of YAP in aggressive glioma subtypes (classical and mesenchymal) and elevated nuclear expression was associated with poor survival (Orr B A, et al. (2011) J Neuropathol Exp Neurol 70: 568-577; and Zhang H, et al. (2016) Tumour Biol).

The Hippo signaling pathway regulates tissue homeostasis and organ size (Yu F X, et al. (2015) Cell 163: 811-828)). Hippo (Mst1/2 in mammals) is activated by the NF2/Merlin-Kibra-Expanded tumor suppressor complex and phosphorylates the large tumor suppressor kinases (Lats1/2) leading to their activation (Yu J, et al. (2010) Dev Cell 18: 288-299). The Lats1/2 kinases phosphorylate YAP resulting in its cytoplasmic retention and degradation (Zhao B, et al. (2010) Genes Dev 24: 72-85). Upstream growth control signals such as cell-cell, cell-matrix or extracellular soluble factors promote YAP and its paralog TAZ to concentrate within the nucleus where they co-activate TEAD transcription factors (Totaro A, et al. (2018) Nat Cell Biol 20: 888-899). Activation of the TEADs (TEAD1-4) initiates expression of cellular communication network (CCN) matricellular protein family, such as connective tissue growth factor (CTGF) and Cyr61 (Mauviel A, et al. (2012) Oncogene 31: 1743-1756; and Piccolo S, et al. (2013) Clin Cancer Res 19: 4925-4930). Expression of these growth factors promotes cell growth, migration, invasive potential and apoptosis avoidance (Piccolo S, et al. (2013) Clin Cancer Res 19: 4925-4930; Hong W, Guan K L (2012) Semin Cell Dev Biol 23: 785-793; and Yu F X, Guan K L (2013) Genes Dev 27: 355-371).

Although evidence exists that YAP promotes tumor growth and metastasis through its interaction with its TEAD interaction domain, direct small molecule inhibitors of this protein-protein interaction (PPI) which are potent and specific are lacking. The identification of these types of molecules is challenging given the large interaction interface and apparent absence of a well-defined druggable binding pocket (Bum-Erdene K, et al. (2019) Cell Chem Biol 26: 378-389).

Described herein are small molecules which block the association of the YAP1 and TEAD1 interaction interface. One of these compounds, NSC682769 was characterized and evaluated for its anti-GBM effects. Biochemical studies demonstrate that NSC682769 binds to YAP, and shown to abrogate YAP-TEAD mediated transactivation and markedly inhibited GBM cell growth, motility and invasiveness. The effects of NSC682769 were also evaluated in GBM cell line xenografts and transgenic (Tg) glioma models.

### Compositions and Pharmaceutical Compositions

Disclosed herein are compounds useful in the methods described herein. In some aspects, the disclosed compounds are capable of inhibiting the binding between Yes-associated protein (YAP) and TEA domain (TEAD), thereby reducing YAP levels or reducing YAP expression. In some aspects, the disclosed compounds are useful in treating cancer. In some aspects, the disclosed compounds are useful in treating glioblastoma. In some aspects, the disclosed compounds are useful in inhibiting tumor growth. In some aspects, the disclosed compounds are useful in inhibiting cell proliferation, cell migration or cell invasiveness. In some aspects, the compound can be:

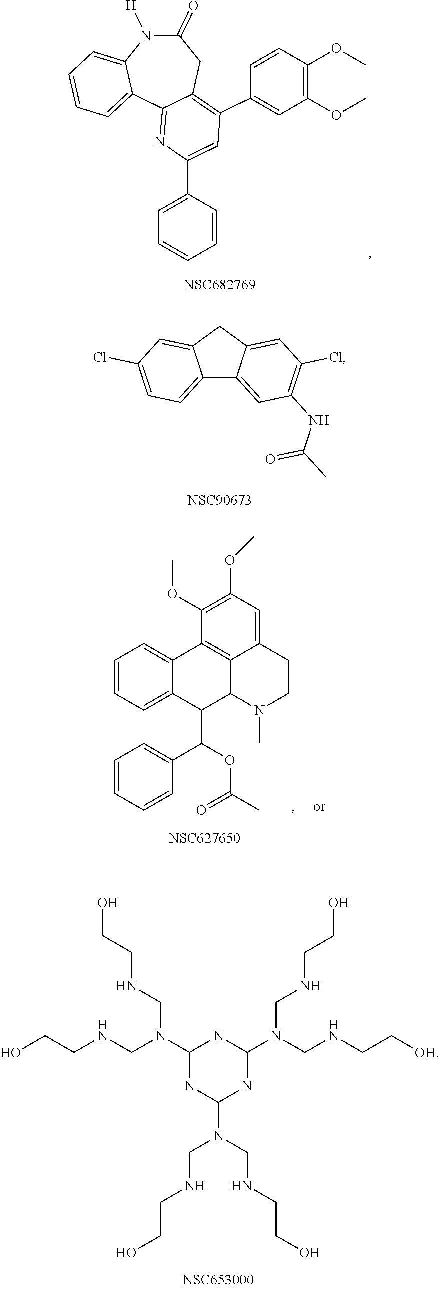 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

In some aspects, the disclosed compound can be

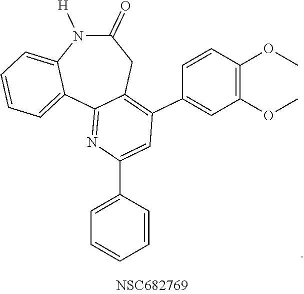 [SMILES: COc1ccc(-c2cc(-c3ccccc3)nc3c2CC(=O)Nc2ccccc2-3)cc1OC]

Also, disclosed herein are pharmaceutical compositions, comprising the compounds or compositions disclosed herein. For example, disclosed herein are pharmaceutical compositions, comprising:

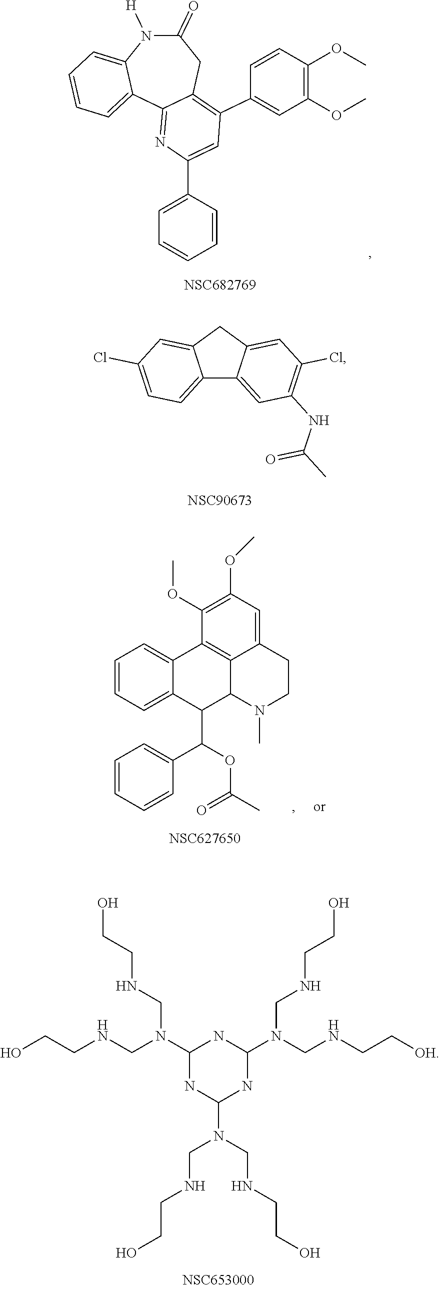 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

Any of the compounds or compositions disclosed herein can further comprise a pharmaceutically acceptable carrier. In some aspects, the pharmaceutical compositions can further comprise a pharmaceutically acceptable carrier.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the compounds or compositions described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions as disclosed herein can be prepared for oral or parenteral administration. Pharmaceutical compositions prepared for parenteral administration include those prepared for intravenous (or intra-arterial), intramuscular, subcutaneous, intrathecal or intraperitoneal administration. Paternal administration can be in the form of a single bolus dose, or may be, for example, by a continuous pump. In some aspects, the compositions can be prepared for parenteral administration that includes dissolving or suspending the disclosed compound in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The compositions disclosed herein can be formulated in a variety of combinations. Disclosed herein are compositions comprising

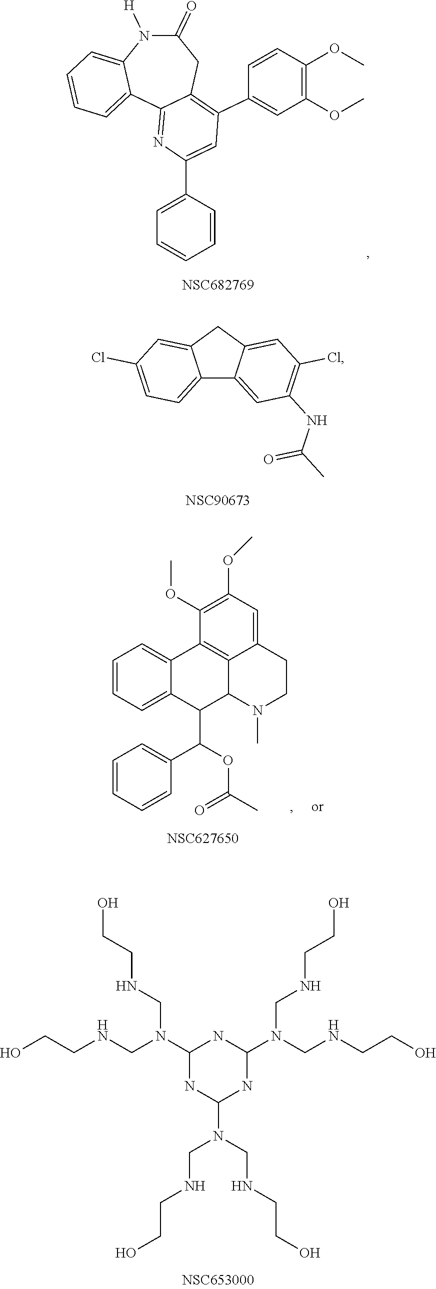 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

and one or more chemotherapeutic agents. The particular combination of the disclosed compound with one or more chemotherapeutic agents (e.g., temzolomide) can vary according to many factors, for example, the particular the type and severity of the cancer. The compositions described herein can be formulated to include a therapeutically effective amount of the disclosed compound alone or in combination with temzolomide. In some aspects, the disclosed compound can be contained within a pharmaceutical formulation. In some aspects, the pharmaceutical formulation can be a unit dosage formulation.

In some aspects, the disclosed compounds can be formulated for oral or parental administration. In some aspects, both the disclosed compounds and the chemotherapeutic agent can be formulated for oral or parenteral administration. In some aspects, the parenteral administration can be intravenous, subcutaneous, intramuscular or direct injection.

In some aspects, the compositions disclosed herein are formulated for oral or parenteral administration. In some aspects, the compositions disclosed herein are formulated for oral, intramuscular, intravenous, subcutaneous, intrathecal, direct injection or intraperitoneal administration.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. The active ingredient can be any of the disclosed compounds described herein in combination with one or more pharmaceutically acceptable carriers. As used herein “pharmaceutically acceptable” means molecules and compositions that do not produce or lead to an untoward reaction (i.e., adverse, negative or allergic reaction) when administered to a subject as intended (i.e., as appropriate).

In some aspects, the route of administration includes but is not limited to direct injection into the brain. Such administration can be done without surgery, or with surgery.

The therapeutically effective amount or dosage of any of the disclosed compounds described herein, and any of the chemotherapeutic agents, used in the methods as disclosed herein, applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

In some aspects, the therapeutically effective dose of any of the chemotherapeutic agents described herein may be less/lower when combined with any of the compounds disclosed herein compared to the dose typically administered in the absence of the compounds disclosed herein. In some aspects, the administration of any of the compounds disclosed herein can increase the efficacy of any of the chemotherapeutic agents described herein.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes. For example, any of the compounds disclosed herein alone or with any of the compounds disclosed herein can be administered in conjunction with standard therapies used to treat cancer (e.g., a chemotherapeutic agent). In some aspects, any of the compounds disclosed herein can be co-formulated with a chemotherapeutic agent. In some aspects, any of the compounds disclosed herein can be co-formulated with a temzolomide.

Any of the compounds or compositions described herein can be administered as a “combination.” It is to be understood that, for example, any of the compounds disclosed herein can be provided to the subject in need, either prior to administration a chemotherapeutic agent or any combination thereof, concomitant with administration of said chemotherapeutic agent or any combination thereof (co-administration) or shortly thereafter.

The dosage to be administered depends on many factors including, for example, the route of administration, the formulation, the severity of the patient's condition/disease, previous treatments, the patient's size, weight, surface area, age, and gender, other drugs being administered, and the overall general health of the patient including the presence or absence of other diseases, disorders or illnesses. Dosage levels can be adjusted using standard empirical methods for optimization known by one skilled in the art. Administrations of the compositions described herein can be single or multiple (e.g., 2- or 3-, 4-, 6-, 8-, 10-, 20-, 50-, 100-, 150-, or more fold). Further, encapsulation of the compositions in a suitable delivery vehicle (e.g., polymeric microparticles or implantable devices) can improve the efficiency of delivery.

### Method of Treatment

The methods disclosed herein can be useful for the treatment of a subject with cancer. In some aspects, the cancer can be glioblastoma. In some aspects, the compounds disclosed herein can inhibit the binding between YAP and TEAD. In some aspects, the compounds disclosed herein can reduce YAP levels in a subject. In some aspects, the compounds disclosed herein can reduce YAP expression in a subject. In some aspects, the compounds disclosed herein can inhibit cell proliferation in a cell, a tissue or a subject. In some aspects, the compounds disclosed herein can inhibit cell migration in a cell, a tissue or a subject. In some aspects, the compounds disclosed herein can inhibit tumor growth in a subject.

Disclosed herein are methods of treating cancer in a subject. In some aspects, the methods comprise: administering to the subject a therapeutically effective amount of compound capable of inhibiting the binding between Yes-associated protein (YAP) and TEA domain (TEAD), thereby reducing YAP levels. In some aspects, the compound capable of inhibiting the binding between Yes-associated protein (YAP) and TEA domain (TEAD) is

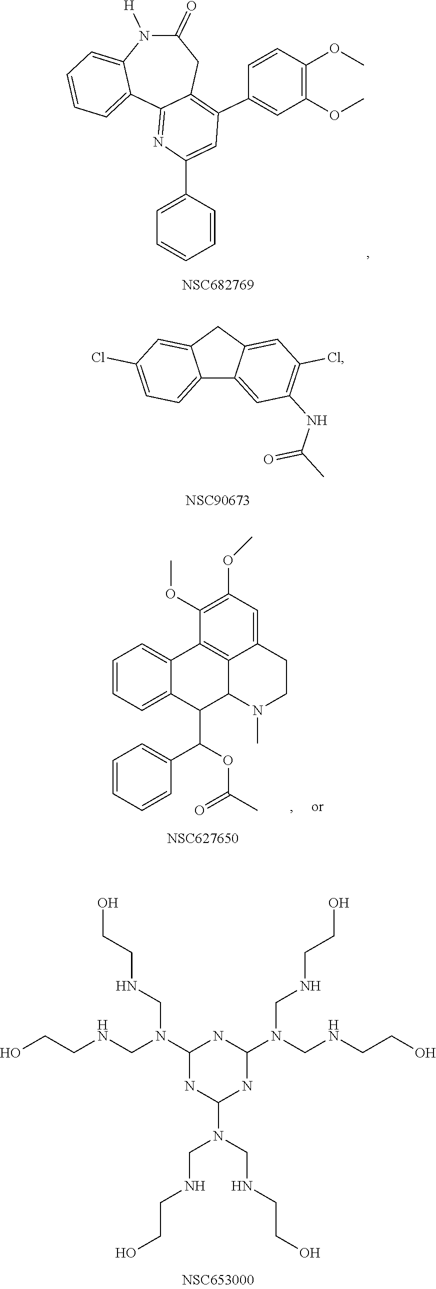 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

Disclosed herein are methods of reducing YAP levels in a subject. In some aspects, the methods comprise: administering to the subject a therapeutically effective amount of compound capable of inhibiting the binding between Yes-associated protein (YAP) and TEA domain (TEAD), thereby reducing YAP levels. In some aspects, the compound capable of inhibiting the binding between Yes-associated protein (YAP) and TEA domain (TEAD) is

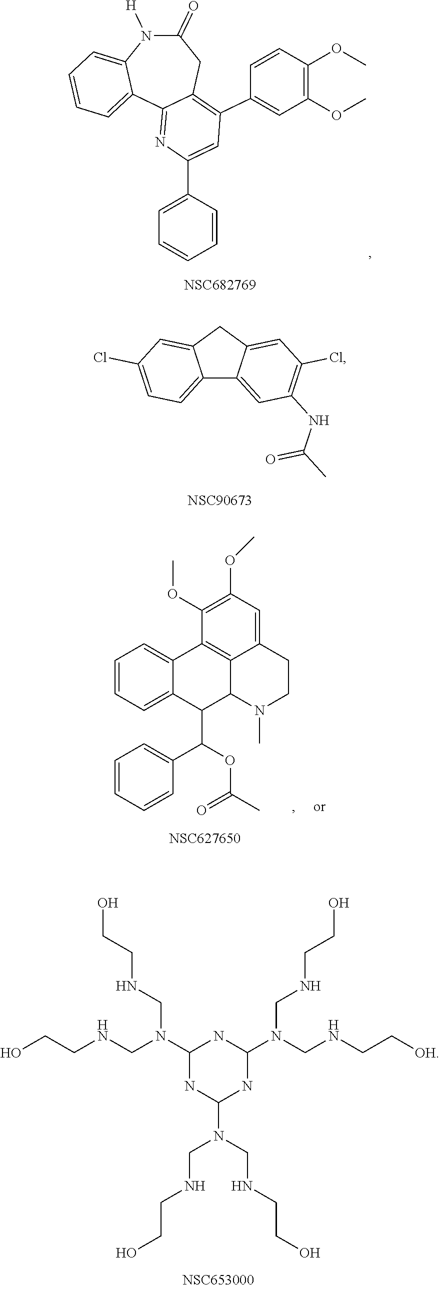 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

Disclosed herein are methods of reducing YAP expression in a subject. In some aspects, the methods comprise: administering to the subject a therapeutically effective amount of compound capable of inhibiting the binding between Yes-associated protein (YAP) and TEA domain (TEAD), thereby reducing YAP expression. In some aspects, the compound capable of inhibiting the binding between Yes-associated protein (YAP) and TEA domain (TEAD) is

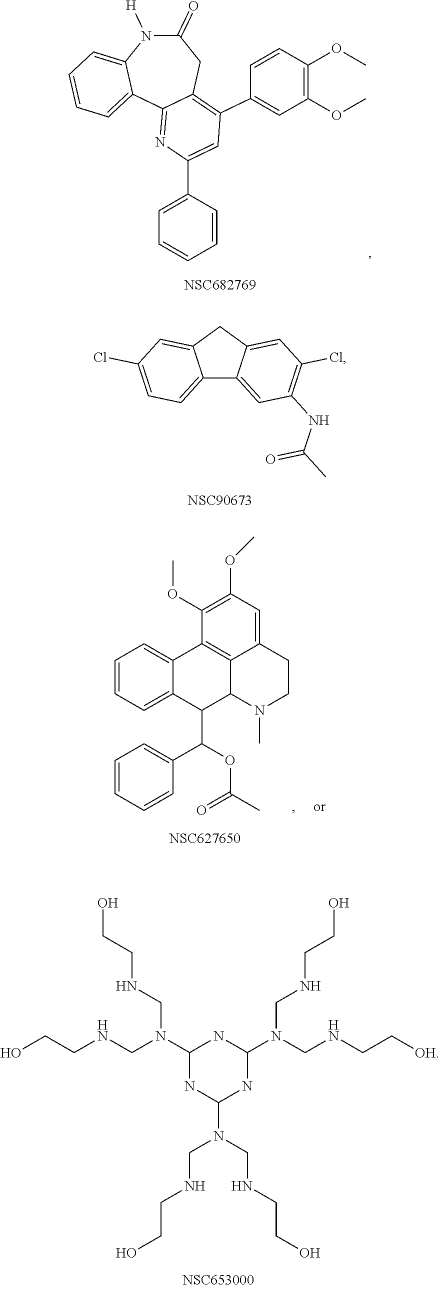 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

Disclosed herein are methods of inhibiting cell proliferation, cell migration, or cell invasiveness. In some aspects, the methods comprise: contacting a cell or tissue or administering to a subject a therapeutically effective amount of compound capable of inhibiting the binding between Yes-associated protein (YAP) and TEA domain (TEAD), thereby reducing YAP levels. In some aspects, the compound capable of inhibiting the binding between Yes-associated protein (YAP) and TEA domain (TEAD) is

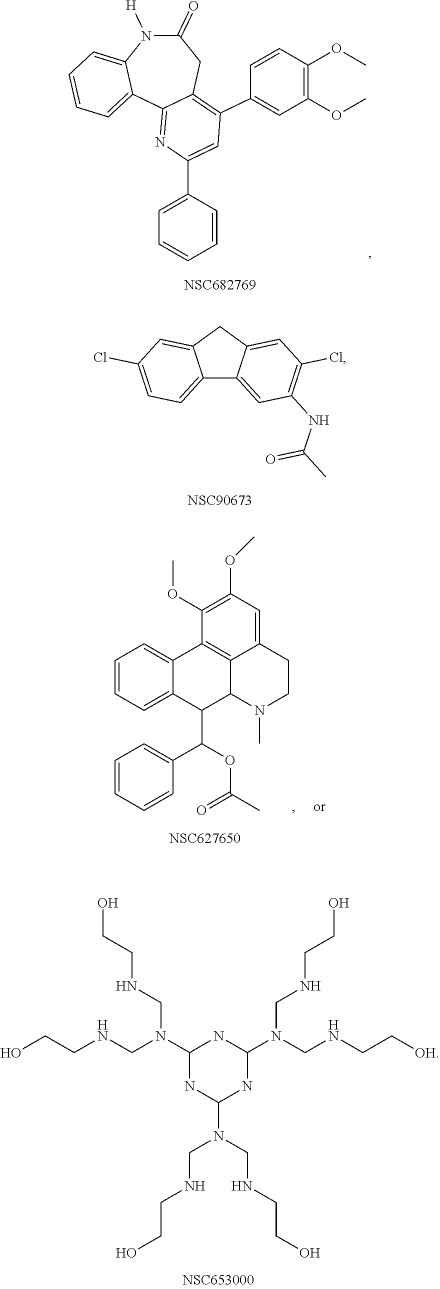 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

Disclosed herein are methods of inhibiting tumor growth. In some aspects, the methods comprise: contacting a cell or tissue or administering to a subject a therapeutically effective amount of compound capable of inhibiting the binding between Yes-associated protein (YAP) and TEA domain (TEAD), thereby reducing YAP levels. In some aspects, the compound capable of inhibiting the binding between Yes-associated protein (YAP) and TEA domain (TEAD) is

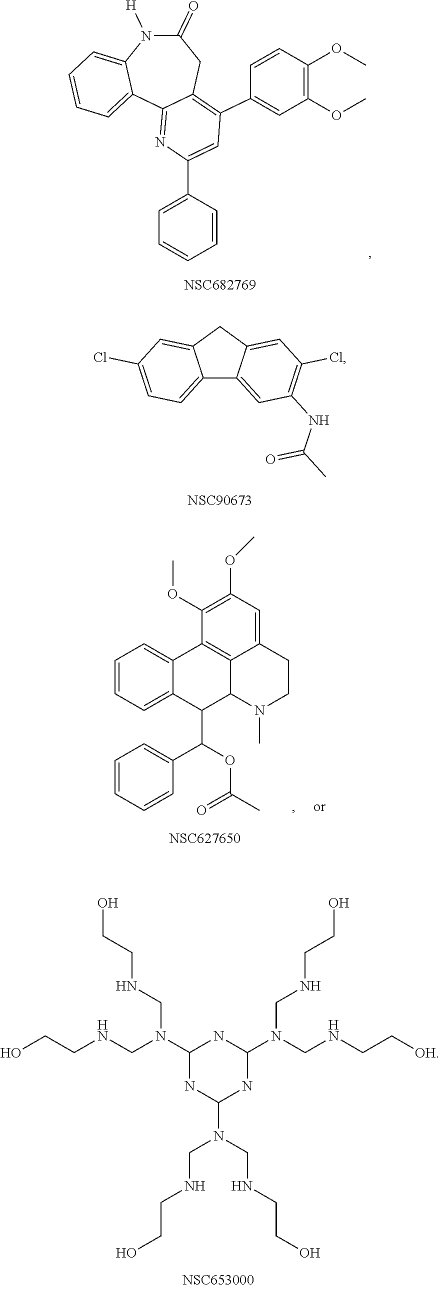 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

Disclosed herein are methods of increasing apoptosis of a cancer cell. In some aspects, the methods comprise: contacting the cancer cell or administering to a subject a therapeutically effective amount of compound capable of inhibiting the binding between Yes-associated protein (YAP) and TEA domain (TEAD), thereby reducing YAP levels. In some aspects, the compound capable of inhibiting the binding between Yes-associated protein (YAP) and TEA domain (TEAD) is

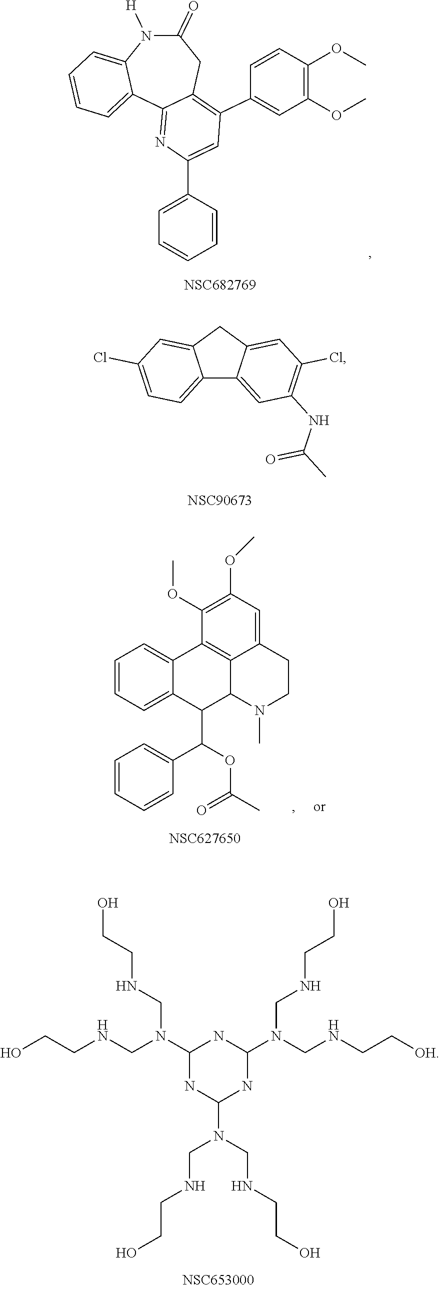 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

In some aspects, the cancer cell can be a glioblastoma cancer cell.

Disclosed herein are methods of treating glioblastoma in a subject. In some aspects, the methods comprise: administering to the subject a therapeutically effective amount of compound capable of inhibiting the binding between Yes-associated protein (YAP) and TEA domain (TEAD), thereby reducing YAP levels. In some aspects, the compound capable of inhibiting the binding between Yes-associated protein (YAP) and TEA domain (TEAD) is

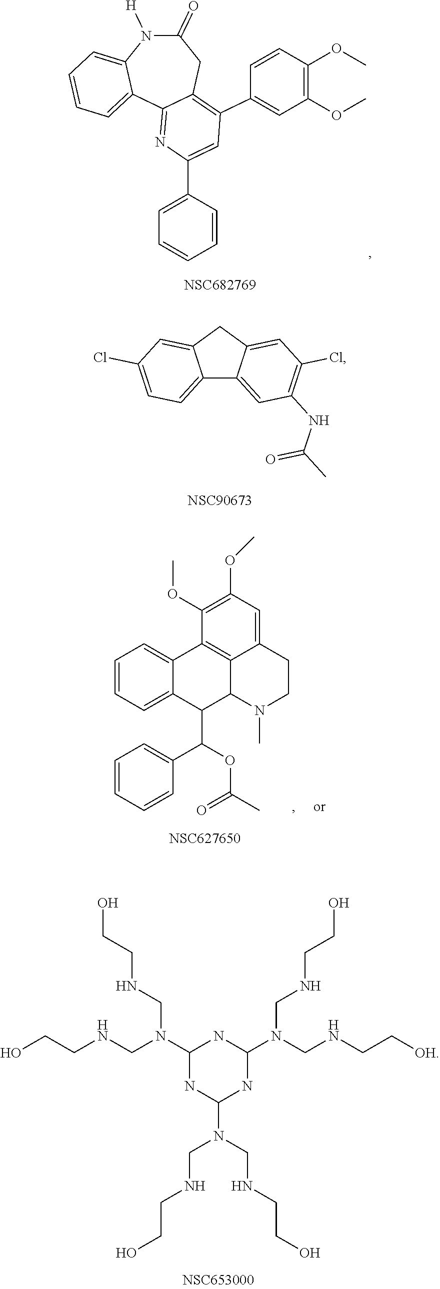 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

In some aspects, the methods also include the step of administering a therapeutic effective amount of any of the compounds disclosed herein. In some aspects, compound can be:

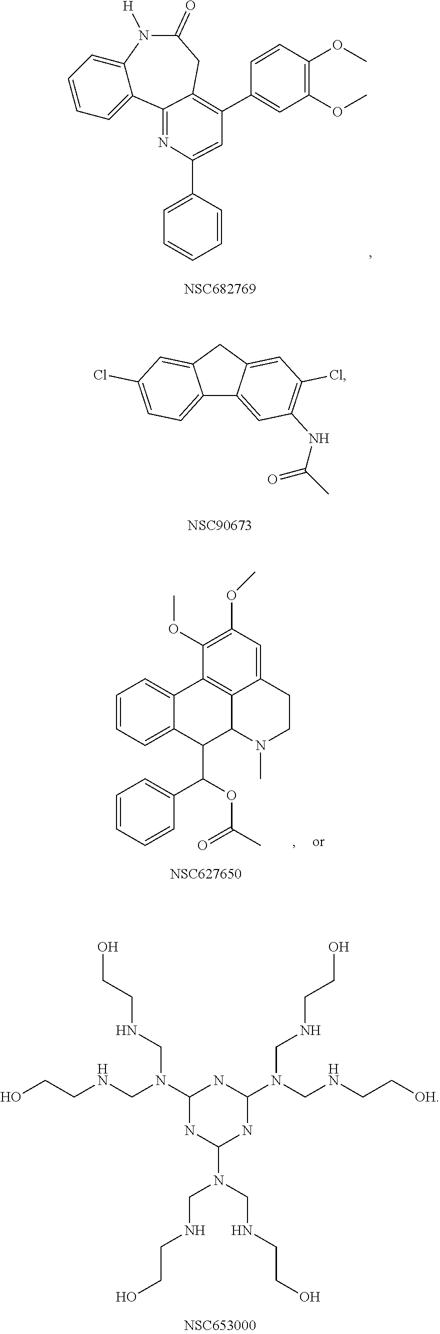 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

In some aspects, the cell can be a vertebrate, a mammalian or a human cell. In some aspects, the cell can be a brain cell. In some aspects, the cell can be a mammalian cell. In some aspects, the mammalian cell can be a brain cell.

In some aspects, the methods can further include the step of identifying a subject (e.g., a human patient) as being in need of treatment before the administration step. In some aspects, the subject has been diagnosed with cancer prior to the administering step. In some aspects, the subject has been identified as having elevated YAP levels. In some aspects, the methods can further include the step of identifying a subject who has cancer or elevated YAP levels and then providing to the subject:

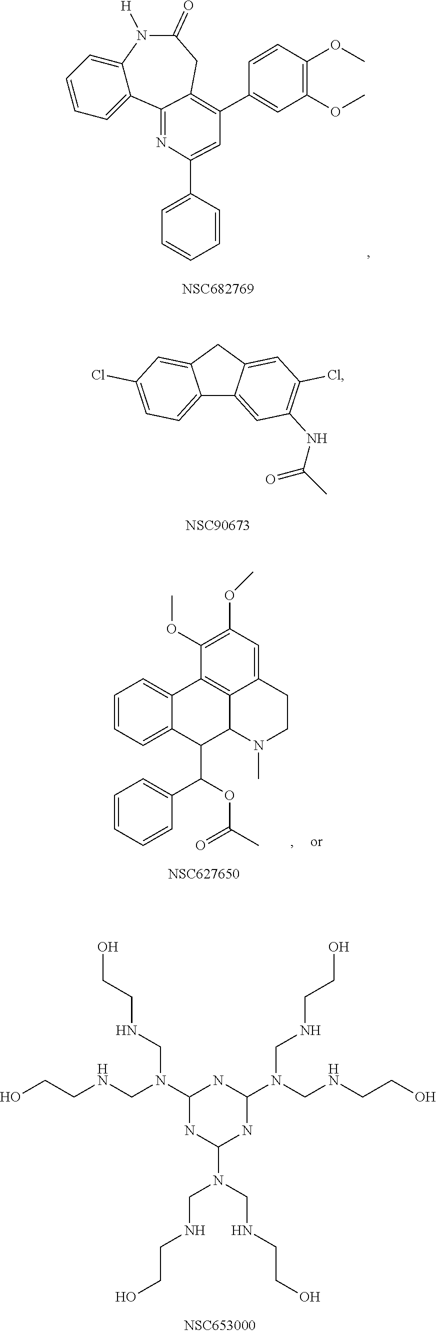 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

or a composition comprising

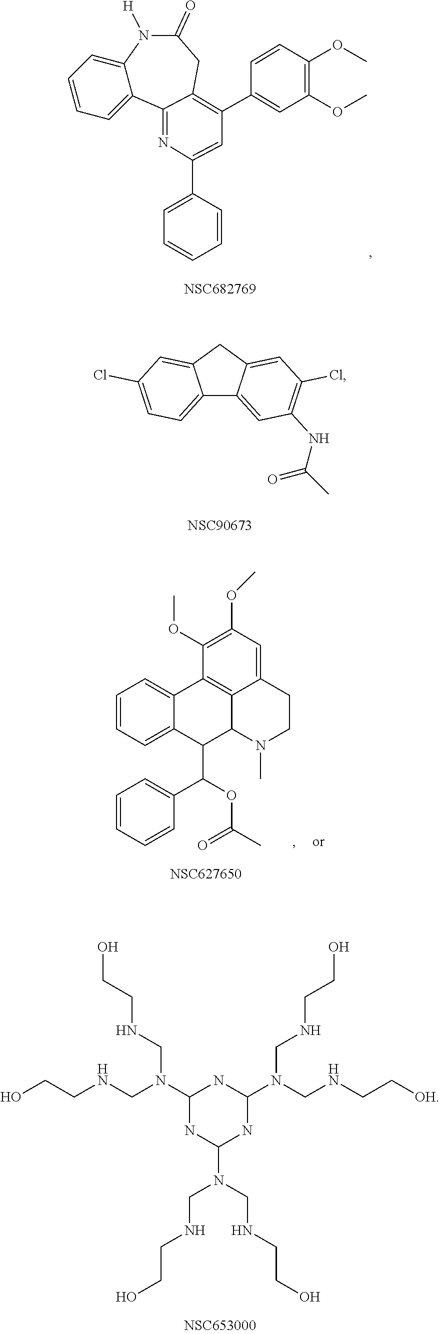 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

In some aspects, the compounds disclosed herein or the composition comprising any of the compounds disclosed herein are capable of inhibiting the binding between Yes-associated protein (YAP) and TEA domain (TEAD) and reducing YAP levels or YAP expression.

In some aspects, the subject has a cancer. In some aspects, the cancer can be a primary or a secondary tumor. In some aspects, the cancer can be a solid tumor. In some aspects, the cancer can be a non-solid tumor. In some aspects, the primary or secondary tumor can be within the subject's brain, breast, pancreas, lung, prostate, liver or thyroid. In some aspects, the cancer can be brain cancer, breast cancer, pancreatic cancer, lung cancer, liver cancer, or thyroid cancer. In some aspects, the cancer can be glioblastoma.

Disclosed herein are methods, comprising: a) obtaining or having obtained a sample comprising tumor cells from a cancer patient; b) measuring the level of Yes-associated protein (YAP) in the tumor cells of the sample; c) identifying the cancer patient as a suitable candidate for treatment with a composition comprising:

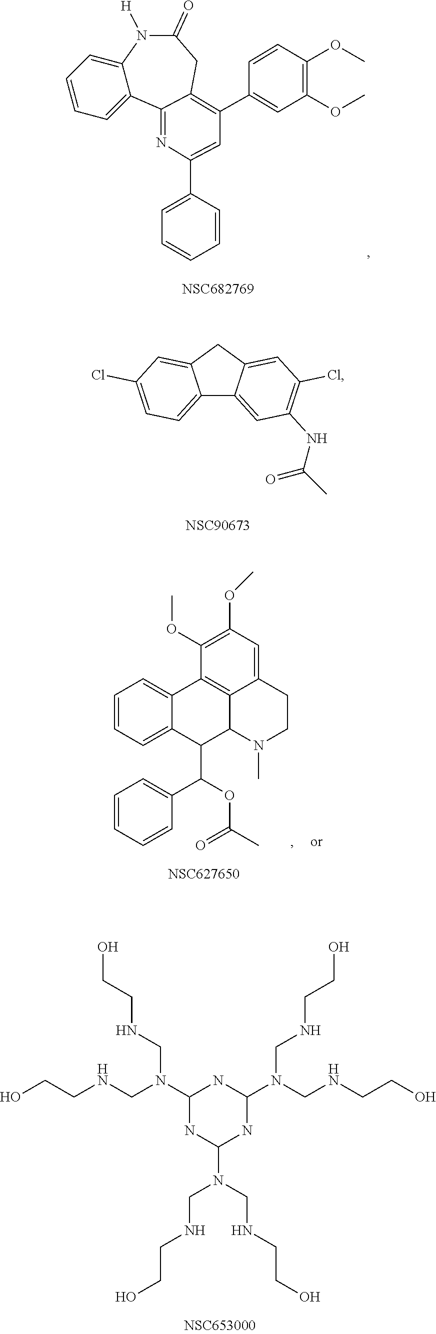 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

when the level of YAP is higher than a level of YAP in a control sample and identifying the cancer patient as an unsuitable candidate for treatment with a composition comprising:

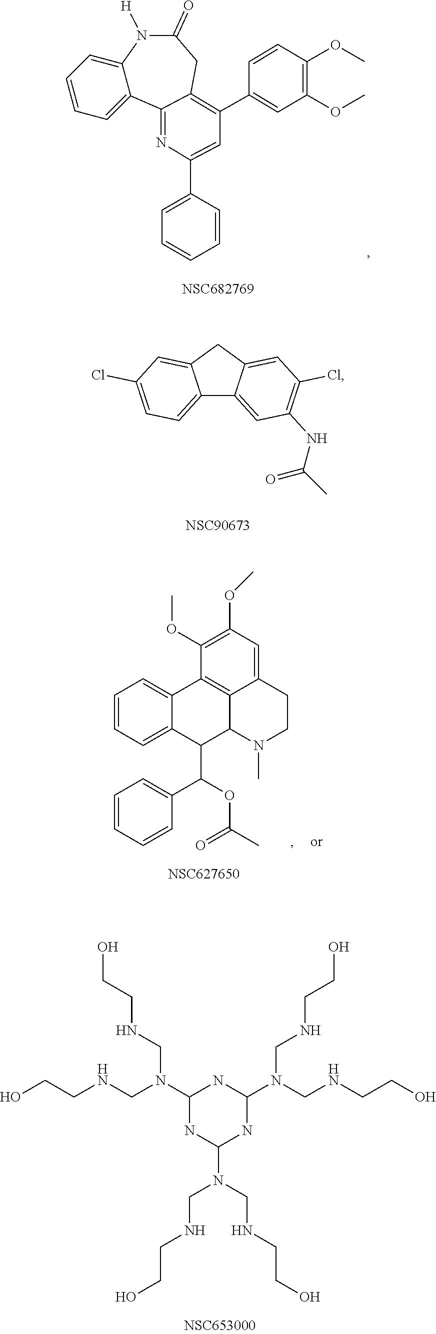 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

when the level of YAP is the same or lower than a level in a control sample; and d) administering the composition comprising

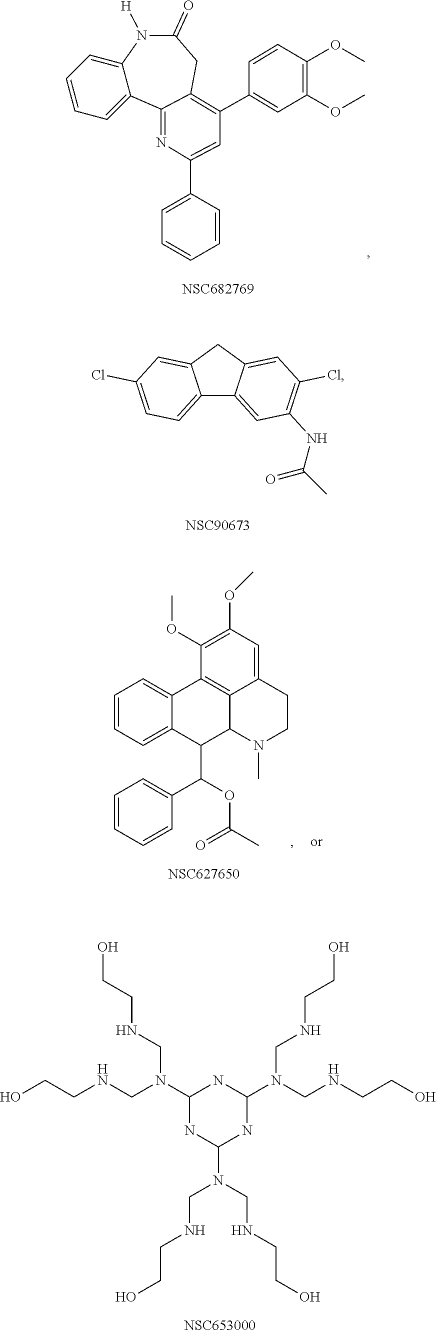 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

to the cancer patient identified as the suitable candidate, and not administering the composition comprising:

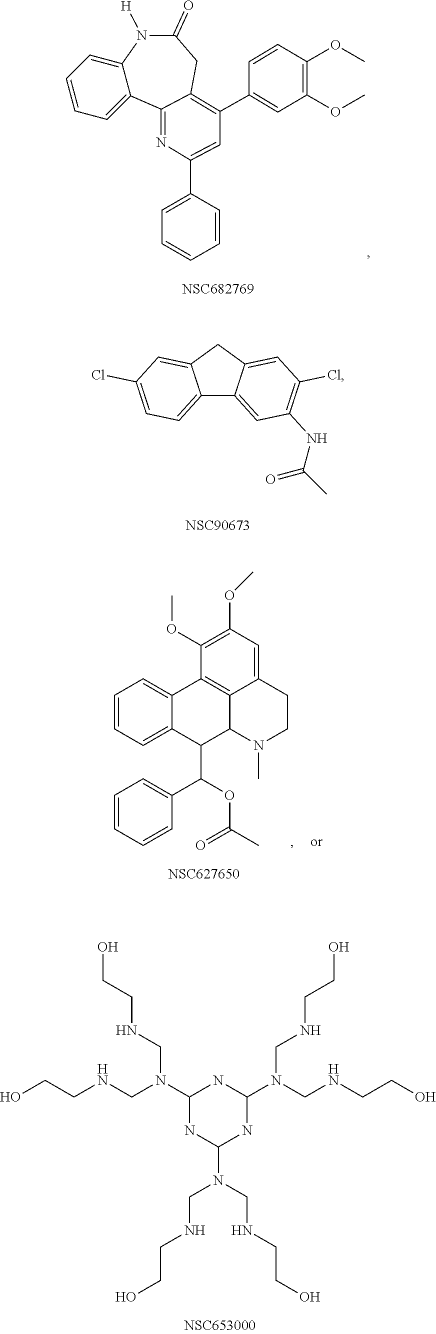 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

to the cancer patient identified as the unsuitable candidate. In some aspects, the tumor cells can be a vertebrate, a mammalian or a human cell. In some aspects, the tumor cell can be a brain cell. In some aspects, the tumor cell can be a mammalian cell. In some aspects, the mammalian cell can be a brain cell. In some aspects, the patient has brain cancer, breast cancer, lung cancer, liver cancer, pancreatic cancer, prostate cancer or thyroid cancer. In some aspects, the brain cancer can be glioblastoma. In some aspects, the sample can be a biopsy. In some aspects, the composition comprising

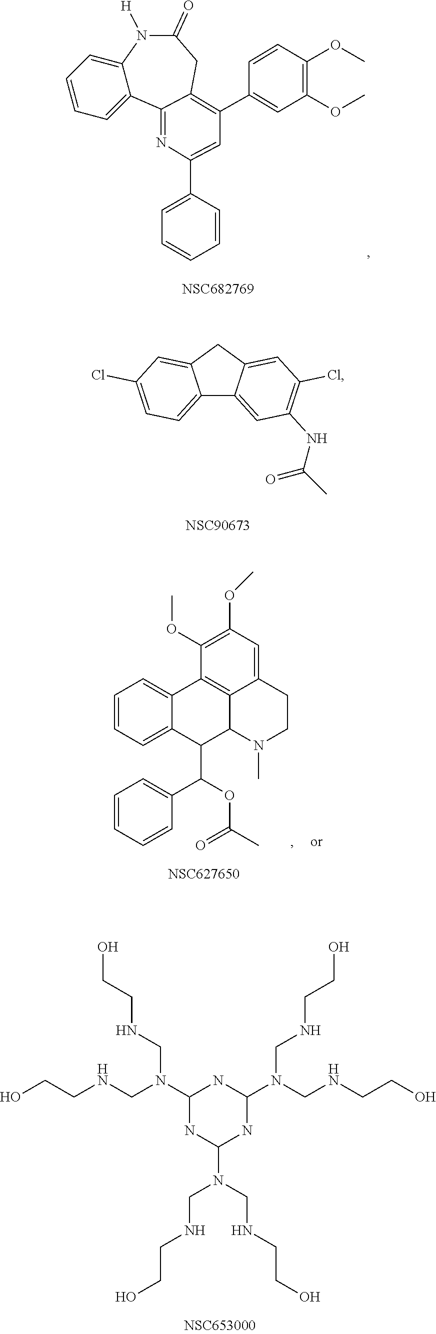 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

can be administered systemically. In some aspects, the method can further comprise administering a therapeutically effective amount of a chemotherapeutic agent. In some aspects, the chemotherapeutic agent can be temozolomide.

The therapeutically effective amount can be the amount of the composition administered to a subject that leads to a full resolution of the symptoms of the condition or disease, a reduction in the severity of the symptoms of the condition or disease, or a slowing of the progression of symptoms of the condition or disease. The methods described herein can also include a monitoring step to optimize dosing. The compositions described herein can be administered as a preventive treatment or to delay or slow the progression of the condition or disease (e.g., cancer).

The compositions disclosed herein can be formulated in a variety of combinations. The particular combination of the compounds or compositions disclosed herein with one or more chemotherapeutic agents (e.g., temozolomide) can vary according to many factors, for example, the particular the type and severity of the cancer. The compositions described herein can be formulated to include a therapeutically effective amount of any of the compounds disclosed herein alone or in combination with a chemotherapeutic agent. In some aspects,

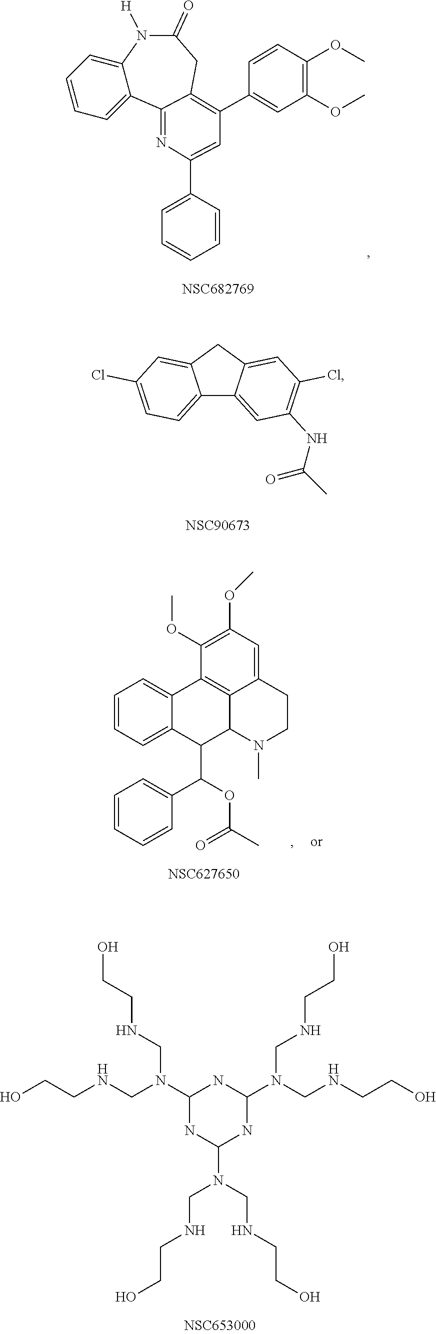 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

can be contained within a pharmaceutical formulation. In some aspects, the pharmaceutical formulation can be a unit dosage formulation.

In some aspects, the methods disclosed herein also include treating a subject having glioblastoma. In some aspects, the methods disclosed herein can include the step of determining YAP levels or expression in a subject. In some aspects, the methods disclosed herein can include the step of determining YAP levels or expression in a tumor in a subject.

In some aspects, the methods described herein can further comprise administering a therapeutically effective amount of a chemotherapeutic agent to the subject. In some aspects, the chemotherapeutic agent can be a DNA damage-inducing agent. In some aspects, the chemotherapeutic agent can be temozolomide.

In some aspects, a chemotherapeutic agent can be, but is not limited to, an alkylating agent, an antimetabolite agent, an antineoplastic antibiotic agent, and a mitotic inhibitor agent.

In a further aspect, the antineoplastic antibiotic agent is selected from doxorubicin, mitoxantrone, bleomycin, daunorubicin, dactinomycin, epirubicin, idarubicin, plicamycin, mitomycin, pentostatin, and valrubicin, or a pharmaceutically acceptable salt thereof.

In a further aspect, the antimetabolite agent is selected from gemcitabine, 5-fluorouracil, capecitabine, hydroxyurea, mercaptopurine, pemetrexed, fludarabine, nelarabine, cladribine, clofarabine, cytarabine, decitabine, pralatrexate, floxuridine, methotrexate, and thioguanine, or a pharmaceutically acceptable salt thereof.

In a further aspect, the alkylating agent is selected from carboplatin, cisplatin, cyclophosphamide, chlorambucil, melphalan, carmustine, busulfan, lomustine, dacarbazine, oxaliplatin, ifosfamide, mechlorethamine, temozolomide, thiotepa, bendamustine, and streptozocin, or a pharmaceutically acceptable salt thereof.

In a further aspect, the mitotic inhibitor agent is selected from irinotecan, topotecan, rubitecan, cabazitaxel, docetaxel, paclitaxel, etopside, vincristine, ixabepilone, vinorelbine, vinblastine, and teniposide, or a pharmaceutically acceptable salt thereof.

### Kits

Disclosed herein are kits that comprise any combination of the compositions described above and suitable instructions (e.g., written and/or provided as audio-, visual-, or audiovisual material). Disclosed herein are kits that comprise any combination of the pharmaceutical compositions described above and suitable instructions (e.g., written and/or provided as audio-, visual-, or audiovisual material). In some aspects, the kit comprises a predetermined amount of a composition or pharmaceutical composition comprising:

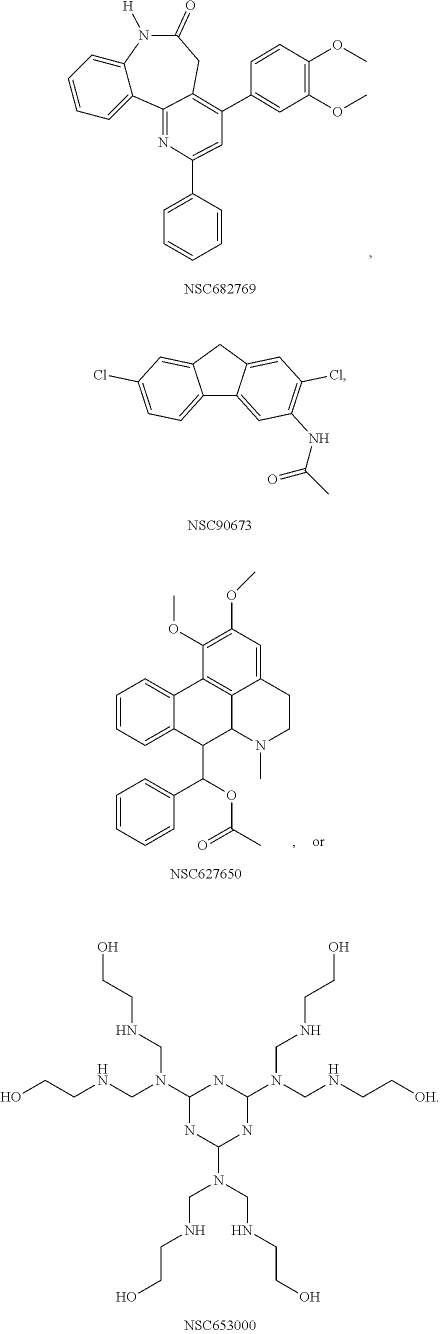 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

The kit can further comprise one or more of the following: instructions, sterile fluid, syringes, a sterile container, delivery devices, and buffers or other control reagents.

### EXAMPLES

### Example 1: Targeting the YAP-TEAD Interaction Interface for Therapeutic Intervention in Glioblastoma

Recent studies have suggested that dysregulated Hippo pathway signaling may contribute to glioblastoma proliferation and invasive characteristics. The downstream effector of the pathway, the Yes-associated protein (YAP) oncoprotein, has emerged as a promising target in glioblastoma multiforme (GBM).

Utilizing a high-throughput yeast two-hybrid based screen, a small molecule was identified which inhibits the association of the co-transcriptional activator YAP1 and the TEA domain family member 1 (TEAD1) transcription factor protein-protein interaction interface. This candidate inhibitor, NSC682769, a benzazepine compound, was evaluated for its ability to affect Hippo/YAP axis signaling and potential anti-glioblastoma properties.

NSC682769 potently blocked association of YAP and TEAD in vitro and in GBM cells treated with submicromolar concentrations. Moreover, inhibitor-coupled bead pull down and surface plasmon resonance analyses demonstrated that NSC682769 binds to YAP. NSC682769 treatment of GBM lines and patient derived cells resulted in downregulation of YAP expression levels resulting in curtailed YAP-TEAD transcriptional activity. In GBM cell models, NSC682769 inhibited proliferation, colony formation, migration, invasiveness and enhanced apoptosis. In tumor xenograft and genetically engineered mouse models (GEMMs), NSC682769 exhibited marked anti-tumor responses and resulted in increased overall survival and displayed significant blood-brain barrier (BBB) penetration.

These results demonstrate that blockade of YAP-TEAD association is a viable therapeutic strategy for glioblastoma.

Materials and Methods. Cell culture, vectors and reagents. Normal mature human neurons were obtained from ScienCell (Carlsbad, CA). Lines were routinely tested to confirm the absence of mycoplasma and authenticated by short tandem repeat profiling (ATCC). The myc-tagged TEAD (pRK5-Myc-TEAD1) and HA-tagged YAP1 (pCl-HA-YAP) vectors were used for mammalian expression and TEAD was subcloned into pGEX-2TK, while YAP was subcloned into pET-14b for bacterial expression and purification. DNA transfections were performed using Effectene transfection reagent according to the manufacturer (QIAGEN) and lentiviral transductions were performed as previously described (Rosenbluh J, et al. (2012) Cell 151: 1457-1473). Large-scale syntheses of NSC682769 was performed by New England Discovery Partners, Branford, CT. The other reagents including kits for ALT, AST and creatinine level determinations were from Sigma-Aldrich.

High-throughput yeast-two-hybrid screening. Screening was carried out as previously described (Benavides-Serrato A, et al. (2017) PLoS One 12: e0176599). Briefly, the yeast two-hybrid strain AH109 modified to maximize the penetration of small molecules, was transformed with constructs containing the minimal required interaction domains of the YAP and TEAD interface based on the co-crystal structure (Li Z, et al. (2010) Genes Dev 24: 235-240). Constructs expressing human YAP1 (residues 50-171 of Accession No. NP_006097) fused to the GAL4 DNA-binding domain (DBD) and TEAD1 (residues 194-411 of Accession No. NP_068780) fused to the GAL4 activation domain (AD) were transformed into the two-hybrid reporter yeast strain. Interaction of YAP and TEAD reconstituted a functional transcription factor capable of inducing GAL4-upstream activating sequence containing reporters and allowed for growth on selective media. Yeast were subsequently interrogated against a >145,000 diversity oriented small molecule library (NCI, DTP) by robotic-aided pinning of compounds onto a lawn of cells. Compounds, which blocked yeast growth, resulted in halo formation and identification of candidate YAP-TEAD inhibitors. As a counterscreen, yeast that were dependent on the interaction of the SV40 large T antigen and p53 for growth on selective media were engineered and compounds which blocked growth of both strains of yeast were considered nonspecific. In three experiments, the Z′ values were between 0.72 and 0.85, consistent with high robustness of the screen.

Protein and mRNA analyses, immunoprecipitation, in vitro bead-coupled inhibitor binding assays and HPLC analyses. Western blot analyses were performed as previously described (Artinian N, et al. (2015) J Biol Chem 290: 19387-19401). Briefly, cells were lysed in RIPA (lysis) buffer containing protease inhibitor cocktail and phosSTOP™ phosphatase inhibitor cocktail (Roche) and extracts resolved by SDS-PAGE. Proteins were transferred to PVDF membranes and incubated with the indicated antibodies. Antigen-antibody complexes were detected using appropriate horseradish peroxidase-conjugated secondary antibodies (GE Healthcare) and enhanced chemiluminescence (Amersham ECL Prime). Primary antibodies for the following proteins were used: actin (no. A5441) was from Sigma-Aldrich, SMAD7 (no. 42-0400) was from ThermoFisher; TBXS (no. ab137833), TEAD2 (no. ab92279) and TEAD4 (no. ab137833) were from Abcam; α-myc-tag (no. 2276S), YAP (no. 12395S), TEAD (no. 13295S), TEAD1 (no. 12292S), TEAD3 (no. 13224S), FOS (no. 4384S), p73 (14620S), α-HA (no. 3724S) and Lamin B2 (no. 12255S) were from Cell Signaling and HSP90 (No. SMC149B) was from StressMarq Biosciences. Immunoprecipitations were performed as previously described (Artinian N, et al. (2015) J Biol Chem 290: 19387-19401). For quantitative RT-PCR, extraction of RNA was performed using Trizol® (Life Technologies). Total RNA was then quantified and integrity assessed using an Agilent 2100 Bioanalyzer (Agilent Technology). Total RNA was reverse transcribed with random primers using the RETROscript™ kit from Invitrogen. SYBR® Green quantitative PCR (Millipore Sigma) was performed in triplicate in 96-well optical plates on an ABI Prism 7000 Sequence Detection System (Life Technologies) according to the manufacturer's instructions. Primer sequences for CTGF and Cyr61 are available upon request. Photo-cross-linked IRES-J007 or NSC682769 beads were prepared as previously described (Holmes B, et al. (2016) J Biol Chem 291: 14146-14159). Briefly, activated Sepharose beads were washed three times with 1 mM aqueous HCl followed by coupling solution (100 mM NaHCO₃ and 50% dioxane mixture). A solution of photoaffinity linker in coupling solution was subsequently added to the beads and incubated at 37° C. for 2 h. After washing five times with coupling solution the beads were blocked and placed in a spin column and washed three times with water and methanol. The beads were subsequently irradiated in a UV cross-linker at 365 nm (4 J/cm²) and washed with methanol. Purified HA-tagged YAP was added to 20 μl of IRES-J007 or NSC682769 cross-linked or control uncross-linked beads. After incubating at 4° C. for 24 h, the beads were washed three times and bound proteins eluted in 10% SDS-PAGE sample buffer at 100° C. for 5 min and the eluted proteins were resolved by SDS-PAGE and immunoblotted using a-HA antibodies. For HPLC analyses, brain samples were homogenized in four volumes of 0.2 M ice-cold perchloric acid containing 3 mM cysteine and centrifuged at 14,000×g for 10 min at 4° C. and the supernatants used for testing. Analysis was performed using an Agilent 1100 HPLC system using a Zorbax Rx-C18 octadecyl silica column (Agilent Technologies). The mobile phase contained 56 mmol/l Na₂HPO₄, 48 mmol/l citric acid, 0.027 mmol/l Na₂EDTA, 0.9 mmol/l octane sulfonic acid sodium and 65:950 acetonitrile/phosphate buffer. The flow rate of the mobile phase was 1 ml/min.

Surface plasmon resonance (SPR), ELISA, YAP-TEAD reporter activity, cellular fractionation, and immunofluorescence analyses. SPR experiments were carried out on a Nicoya OpenSPR™ instrument using immobilized YAP on a carboxyl-coated sensor. Binding was observed as the change in response units (RU) as analyte was injected at a flow rate of 10 μl/min at 25° C. TraceDrawer™ was used for kinetic analyses. The PathScan® total-YAP ELISA kit was obtained from Cell Signaling and used according to the manufacturer's instructions. YAP-TEAD reporter activity was determined as previously described (Kim N G, Gumbiner B M (2015) J Cell Biol 210: 503-515). HOP-flash contains multiple copies of wild-type TEAD-binding sites with a minimal promoter upstream of a luciferase reporter gene. Luciferase activity was measured via a luciferase assay system (Promega). Nuclear-cytoplasmic fractionation was performed according to Dignam et al. (Dignam J D, et al. (1983) Nucleic Acids Res 11: 1475-1489). Briefly, the buffers used were kept on ice and centrifugations were done at 4° C. with soft braking. After a single wash with PBS, cells were scraped with PBS (containing 1 mM DTT and 1× protease inhibitor) and harvested by centrifugation at 1000×g for 15 min. The cell pellet was gently resuspended with five times the volume of pellet with buffer A (10 mM HEPES, pH 7.9, 1.5 mM MgCl₂, 10 mM KCl, 0.5 mM DTT, and 1× protease inhibitor) and incubated on ice for 15 min, followed by homogenization (Wheaton). Cell lysis was monitored by trypan blue staining. The cell lysate was spun at 1000×g for 5 min to collect the pellet as the nuclear fraction and the supernatant as the cytoplasmic fraction. For immunofluorescence staining, cells were grown on coverslips and were fixed with 4% paraformaldehyde in PBS for 15 min at room temperature (or overnight at 4° C.) and washed three times for 5 min in 100 mM glycine containing PBS, followed by permeabilization with 0.1% Triton X-100 in PBS for 10 min. After blocking with 3% nonfat dry milk in PBS for 1 h, cells were incubated with primary antibody diluted in 1% BSA/PBS overnight at 4° C. After washing with PBS, cells were incubated with Alexa Fluor 488- or 594-conjugated secondary antibodies (Invitrogen) for 1 h and washed with PBS. Cell nuclei were counterstained and mounted with a mounting medium with DAPI (Vectashield; Vector Laboratories). Immunofluorescence staining images were collected at room temperature on a Zeiss Axio Imager M2 microscope coupled to a cooled digital CCD camera (ORCA®-R² C10600-10B-H; Hamamatsu Photonics).

Cell proliferation, clonogenic, migration, invasion and apoptosis assays. Cell proliferation was determined via Cell Titer-Glo® luminescent cell assays (Promega). Clonogenic assays were done by plating a total of 1,000 cells per well in 24-well plates in a total volume of 400 μL using a two-layered soft agar system as previously described (Masri J, et al. (2007) Cancer Res 67: 11712-11720). Cell migration assays were conducted using precoated modified Boyden chambers as previously described (Benavides-Serrato A, et al. (2017) PLoS One 12: e0176599). For invasion assays through Matrigel®, 20,000 cells were loaded in the top well of Boyden chambers that contained growth factor-reduced Matrigel® extracellular basement membrane over a polyethylene terephthalate membrane with 8-mm pores (BD Biosciences). For apoptosis determinations, cells were stained using a FITC-conjugated annexin V/PI and subjected to flow cytometry (Annexin V-FITC Early Apoptosis Detection kit, Cell Signaling). TUNEL staining of tumor sections was performed using the TACSXL DAB In Situ Apoptosis Detection kit (Trevigen) according to the manufacturer's instructions.

Animal studies. Xenografts of LN229 cells were performed in 4-6-week-old female C.B.-17-scid (Taconic) mice as previously described (Holmes B, et al. (2016) J Biol Chem 291: 14146-14159). Tumors were harvested at autopsy for immunoblot analysis. Sections of paraffin-embedded tumors on slides were processed for immunohistochemistry as previously described (Benavides-Serrato A, et al. (2017) PLoS One 12: e0176599). For GEMM studies, double transgenic GFAP-EGFRvIII; GFAP-Cre/Rictor^{loxP/loxP} mice as previously described were used (Bashir T, et al. (2012) PLoS One 7: e47741). Determinations of blood plasma and brain concentrations of NSC682769 following IV administration were performed in C57/BL6 mice.

Statistical analyses. Statistics were performed with Student's t-tests and ANOVA models using Systat 13 (Systat Software, Chicago, IL, USA). P-values of less than 0.05 were considered significant.

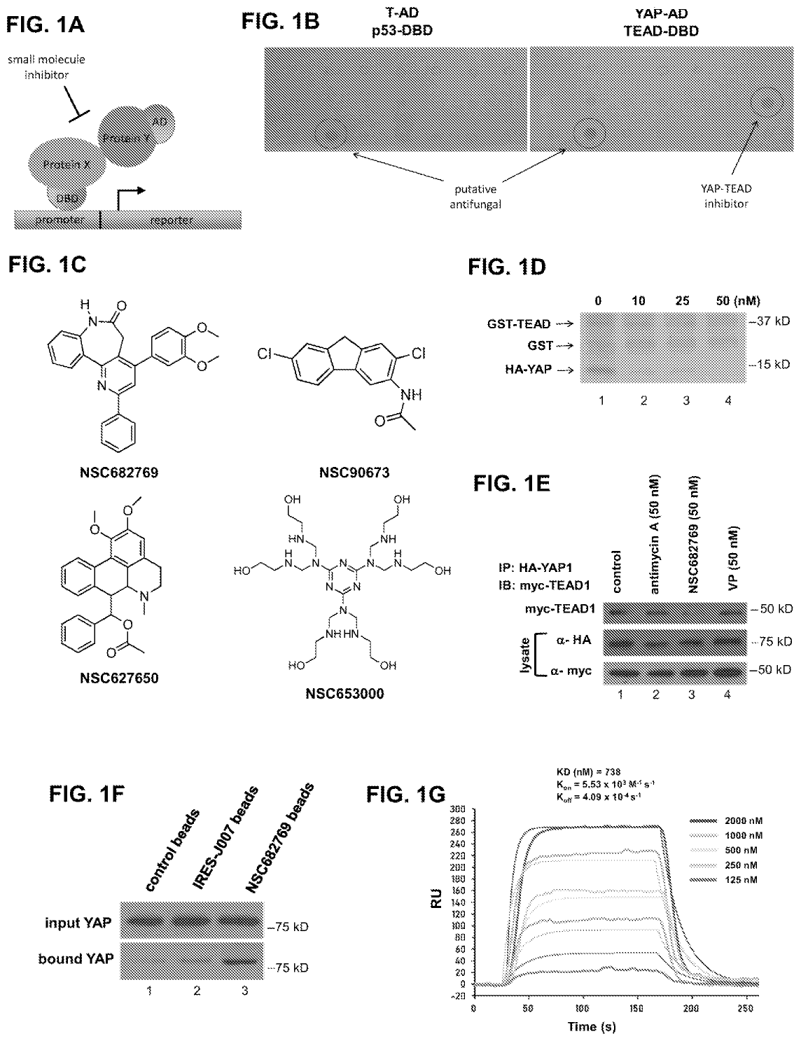

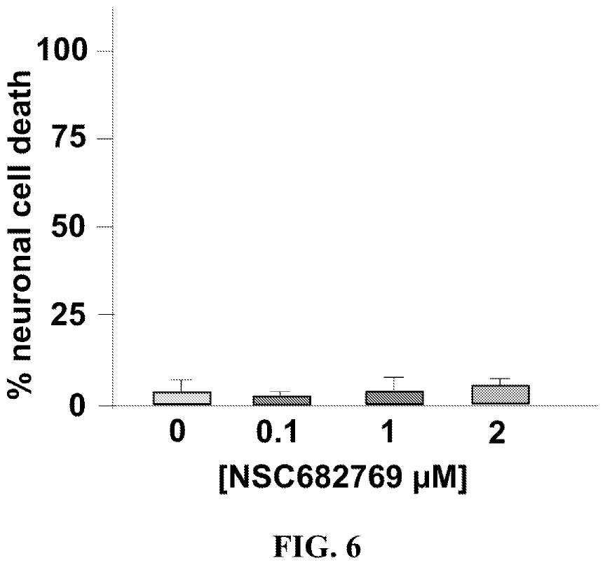

Results. Identification of small molecule inhibitors of the YAP-TEAD interface interaction. To identify small molecule inhibitors of YAP-TEAD association, a yeast two-hybrid screen was engineered to identify compounds which blocked the YAP1-TEAD1 interface interaction (Gibault F, et al. (2018) Cancers (Basel) 10: 140; and Kato-Stankiewicz J, et al. (2002) Proc Natl Acad Sci USA 99: 14398-14403). Yeast which were dependent on the interaction of these two protein domains for growth under selective conditions were used to screen the NCl/DTP small molecule library (FIG. 1A). Shown in FIG. 1B, compounds that blocked growth under these conditions were identified and counterscreened for inhibition of growth of a strain dependent on the SV40 large T-antigen and p53 interaction. Compounds which inhibited both interactions were omitted and presumed to be generally toxic. Additionally, some compounds were predicted to be toxic to mammalian cells based on their structure and not pursued. NSC682769 (FIG. 1C) was selected to study as it exhibited minimal toxicity to normal human neurons (FIG. 6). NSC682769 demonstrated marked inhibition of His-tagged YAP1 (residues 50-171 of Accession No. NP_006097) and GST-TEAD1 (residues 194-411 of Accession No. NP_068780) association in an in vitro GST-pull down assay (FIG. 1D) in a concentration-dependent manner. Moreover, NSC682769 blocked the association of YAP1 with TEAD1 in immunoprecipitates of YAP1 from LN229 GBM cells treated with the inhibitor (FIG. 1E). To examine the mechanism by which NSC682769 inhibits YAP, the ability of full-length human YAP1 to bind to NSC682769 cross-linked affinity beads prepared using a photo-cross-linking procedure (Holmes B, et al. (2016) J Biol Chem 291: 14146-14159) was determined. Recombinant YAP was incubated with control, IRES-J007 (negative control), or NSC682769-coupled beads and binding analyzed by immunoblotting for YAP (FIG. 1F). As observed, native YAP associated strongly with NSC682769 while no significant binding was observed in control uncoupled or IRES-J007 coupled beads. Finally, the binding of NSC682769 to immobilized YAP was examined using SPR analyses. As shown in FIG. 1G, NSC682769 bound YAP in a concentration-dependent manner and reached equilibrium rapidly. The K_{d} was determined from steady-state binding associations and calculated at 738 nM supporting a direct interaction between NSC682769 and YAP. These results demonstrate that NSC682769 directly binds to YAP blocking its association with TEAD.

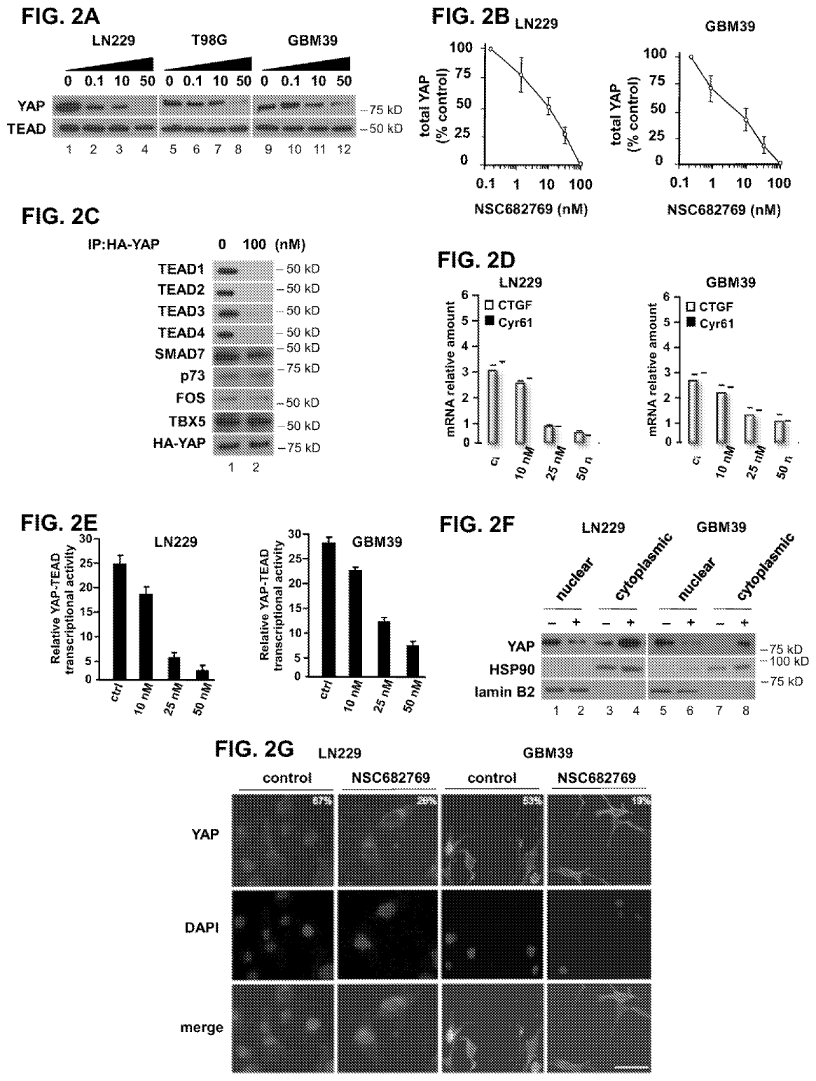

NSC682769 inhibits YAP expression, YAP-dependent transcriptional activity and results in cytoplasmic localization in GBM. To examine the effects of NSC682769 on YAP activity, GBM lines LN229, T98G and the patient-derived line GBM39 were treated and YAP expression levels were examined. As shown in FIG. 2A, following an 18 h exposure with increasing concentrations of NSC682769, YAP levels decreased while TEAD levels were unaffected. The inhibition of YAP expression by NSC682769 was also evaluated by ELISA in LN299 and GBM39 cells (FIG. 2B). The calculated IC₅₀'s for YAP were 11.8 nmol/L in LN229 cells and 5.1 nmol/L in the patient-derived GBM39 cells. It was then examined whether NSC682769 would inhibit the association of YAP with particular TEAD family members (TEAD1-4). LN229 cells stably expressing an HA-tagged YAP were treated with NSC682769 for 18 h and lysates immunoprecipitated with α-HA antibodies and immunoblotted for TEADs 1-4 and several other transcription factors reported to associate with YAP (Strano S, et al. (2001) J Biol Chem 276: 15164-15173; Ferrigno O, et al. (2002) Oncogene 21: 4879-4884; Rosenbluh J, et al. (2012) Cell 151: 1457-1473; and Shao D D, et al. (2014) Cell 158: 171-184). As shown in FIG. 2C, NSC682769 treatment inhibited the binding of the four TEAD members to YAP, while having no appreciable effects on SMAD7, p73, FOS or TBXS association. The expression of two YAP-target genes, CTGF and Cyr61 were monitored. Both of these transcripts were downregulated following exposure to NSC682769 in LN229 and GBM39 cells (FIG. 2D). To assess the effects of NSC682769 on YAP transcriptional activity, LN229 or GBM39 cells were transfected with a TEAD binding site-based reporter (HOP-flash) and treated with increasing concentrations of the inhibitor. As shown in FIG. 2E, YAP-TEAD transcriptional activity was markedly reduced in response to NSC682769. To determine if NSC682769 altered the cellular localization of YAP, cell fractionation experiments were performed. LN229 or GBM39 cell were treated with NSC682769 and cytoplasmic and nuclear extracts immunoblotted for YAP. As shown in FIG. 2F, exposure of either LN229 or GBM39 cells to NSC682769 (50 nM, 6 h) resulted in a reduction of nuclear YAP and a concomitant accumulation of cytoplasmic YAP consistent with its marked reduction in expression at longer time points following exposure to the inhibitor. These results were confirmed in a series of immunofluorescence microscopy experiments in which either LN229 or GBM39 cells were treated with NSC682769 (50 nM, 6 h) and YAP localization determined (FIG. 2G). As shown, in LN229 and GBM39 control untreated cells the amount of nuclear staining was determined to be 67% and 53%, respectively. However, in cells treated with NSC682769 the relative amount of nuclear YAP was reduced to 26% and 19%, respectively. Together these data show that NSC682769 inhibits expression, activity and results in cytoplasmic redistribution of YAP in GBM cells.

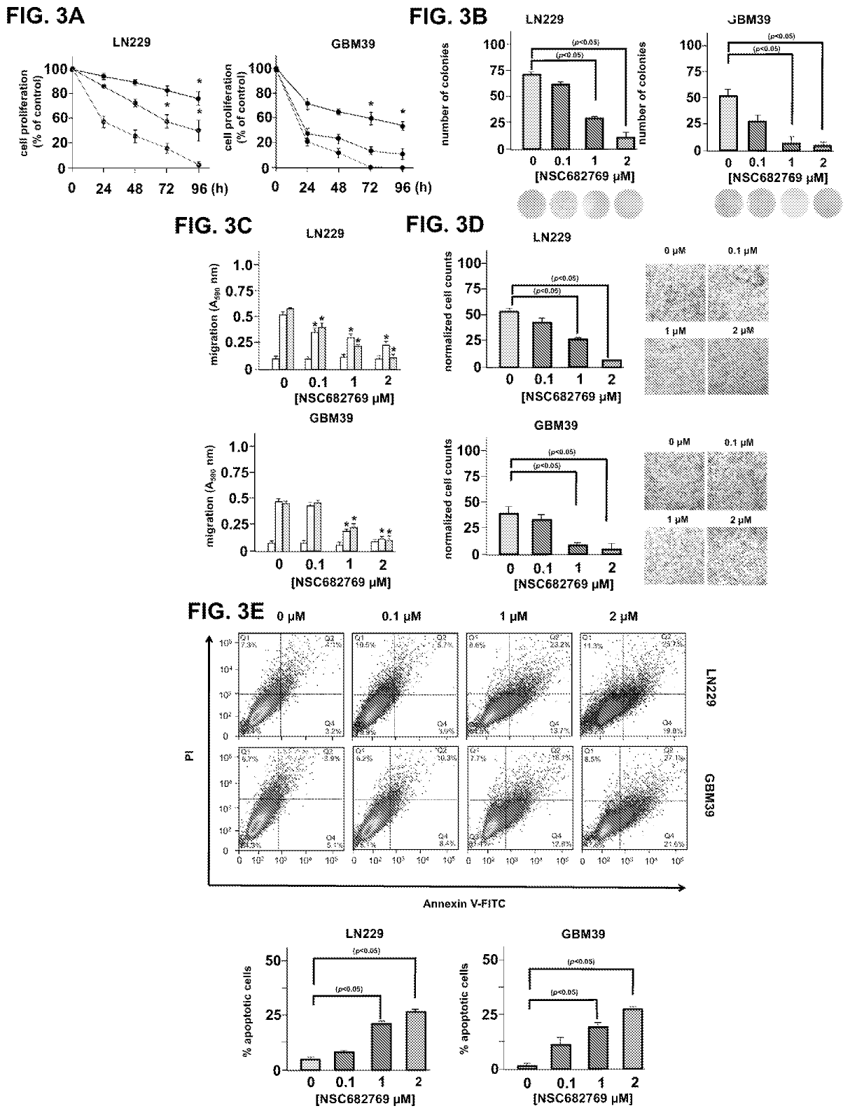

NSC682769 inhibits GBM proliferation, migration and invasive characteristics. To examine the anti-GBM properties of NSC682769, its effects on cell proliferation were determined. As shown in FIG. 3A, NSC682769 significantly inhibited the growth of LN229 and patient-derived GBM39 cells. To determine whether NSC682769 inhibited anchorage-independent growth, soft agar colony formation assays were performed. As shown in FIG. 3B, clonogenic growth of both LN229 and GBM39 cells was inhibited by NSC682769. To determine if NSC682769 would affect cell migration, the capacity of treated cells to cross a vitronectin or fibronectin-coated Boyden chambers relative to chambers coated with control BSA were determined. NSC682769 inhibited the number of cells capable of migrating towards vitronectin or fibronectin-coated surfaces relative to control BSA-coated chambers (FIG. 3C). The ability of NSC682769 to inhibit GBM cell invasiveness was examined in Matrigel invasion assays. As shown in FIG. 3D, the inhibitor reduced the number of cells able to traverse Matrigel-coated membranes. Additionally, apoptosis was enhanced in LN229 and GBM39 cells treated with NSC682769 (FIG. 3E). These data demonstrate the significant anti-GBM effects of NSC682769 in vitro.

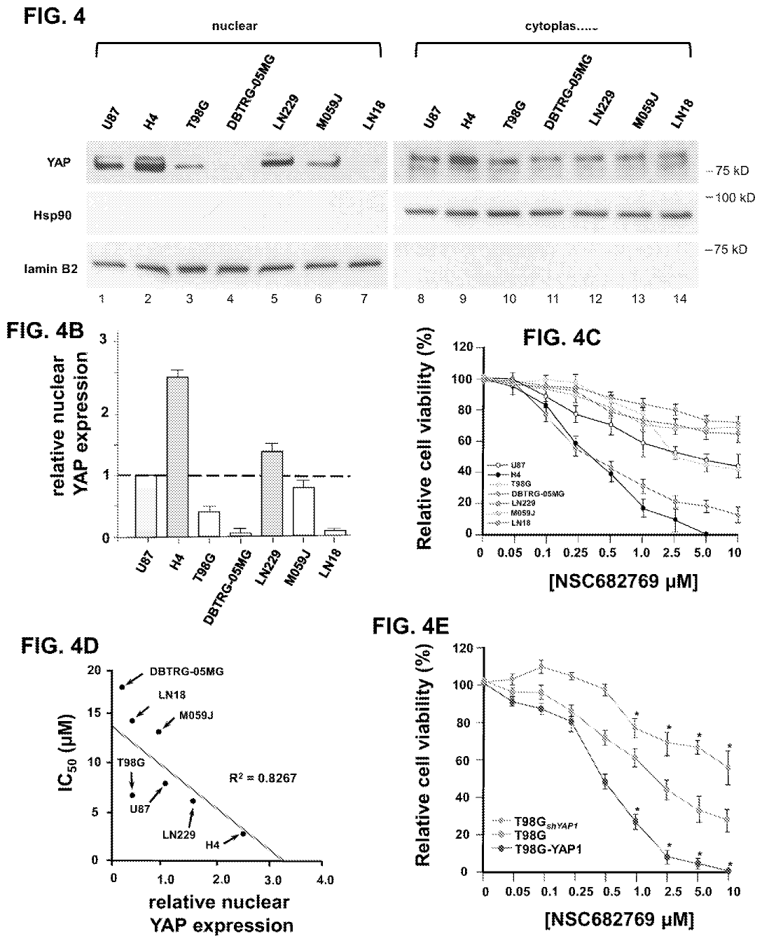

Increased YAP expression directly correlates with NSC682769 sensitivity in GBM cells. The relative expression of YAP is increased in gliomas and promotes tumor proliferation via enhanced YAP-TEAD interactions and promotion of YAP-target gene transcription (Orr B A, et al. (2011) J Neuropathol Exp Neurol 70: 568-577; Liu M, et al. (2017) Lab Invest 97: 1354-1363; and Zhang H, et al. (2016) Tumour Biol.). To examine whether differences in YAP expression may alter sensitivity to NSC682769, the relative expression of nuclear versus cytoplasmic YAP was determined in a panel of GBM lines (FIG. 4A). Expression of nuclear YAP varied greatly and was normalized to levels displayed in U87 cells. Cytoplasmic levels of YAP were more uniform, although the H4 line harbored elevated levels of both nuclear and cytoplasmic YAP relative to U87 cells. The expression of nuclear YAP between the GBM lines is shown in FIG. 4B. H4 and LN229 cells displayed the highest levels of nuclear YAP, while the T98G, DBTRG-05MG and LN18 lines expressed low levels and U87 and M059J lines expressed intermediate levels. Cell viability was then determined using ATP-release assays on these lines over a wide range of NSC682769 concentrations and IC₅₀s calculated (FIG. 4C). As shown in FIG. 4D, a significant inverse relationship was observed between the IC₅₀ for NSC682769 and the relative nuclear YAP expression for these lines. These data demonstrated that lines with elevated nuclear YAP expression were the most sensitive to the compound while those with low levels were relatively resistant. T98G cells were stably transfected with either an shRNA plasmid targeting YAP1 or with a mammalian expression construct to overexpress YAP1. These cells were also examined for NSC682769 sensitivity and as shown in FIG. 4E, cells in which YAP1 had been knocked down were relatively resistant to NSC682769 compared to the parental T98G line, however, cells in which YAP1 was overexpressed were significantly more sensitive.

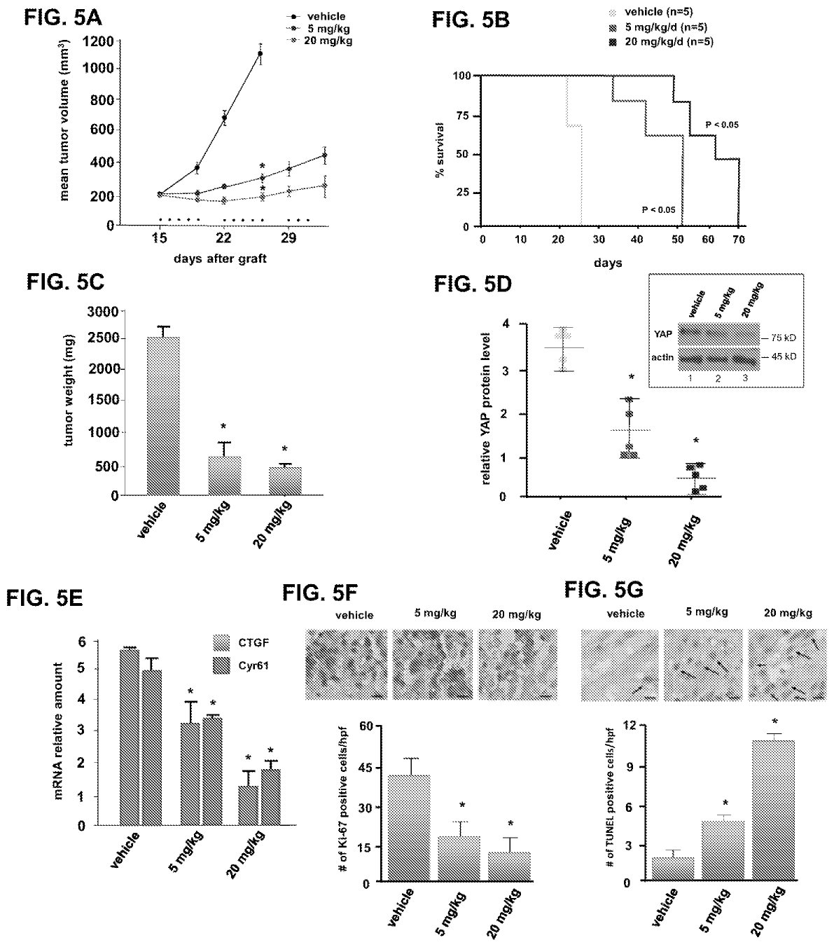

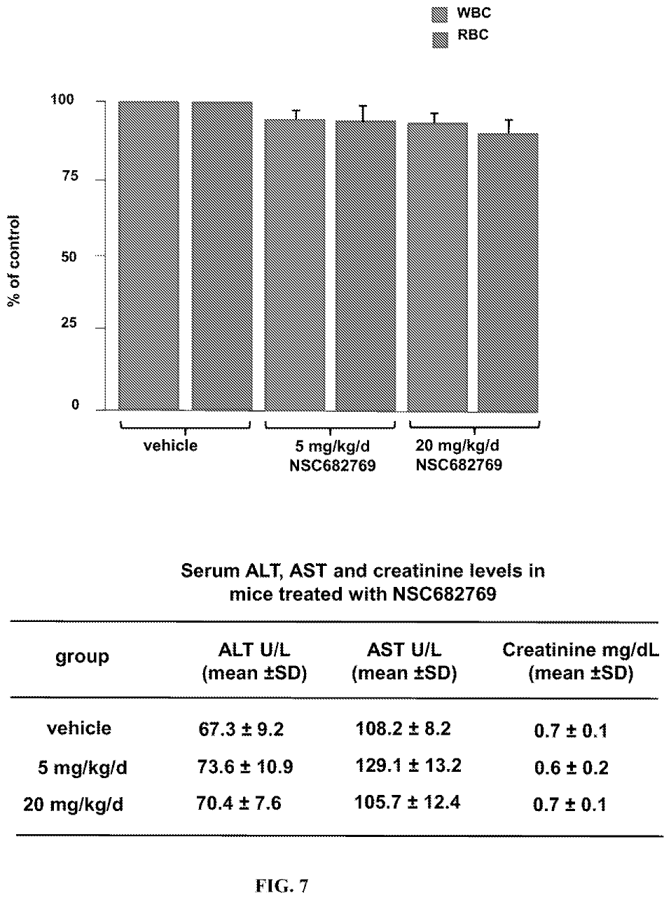

NSC682769 inhibits GBM tumor growth in xenografted mice. To examine the effects of NSC682769 in vivo, the ability of the compound to inhibit the growth of LN229 xenografted tumors implanted subcutaneously in SCID mice was tested. Following implantation, when tumors reached ˜200 mm³, mice were randomized into treatment groups receiving vehicle, 5 mg/kg/d and 20 mg/kg/d of NSC682769. As shown in FIG. 5A, mice undergoing therapy with NSC682769 at either dosing schedule displayed a significant reduction in tumor growth rate relative to mice receiving vehicle (5 mg/kg/d; 66% inhibition at end of dosing period and tumor growth delay 11 days; 20 mg/kg/d; 83% inhibition at end of dosing period and tumor growth delay of 16 days). Overall survival of mice at either dosing regimen of NSC682769 was markedly extended relative to vehicle treated mice (FIG. 5B). No overt short or long-term toxicity or weight loss associated with either dosing schedule in the mice was observed. Moreover, neither blood cell counts nor serum alanine aminotransferase (ALT), aspartate aminotransferase (AST) or creatinine levels were affected by NSC682769 (FIG. 7). A significant reduction in harvested tumor weights was observed from both dosing regimens as compared to vehicle as shown in FIG. 5C. The relative expression level of YAP was reduced in harvested tumors from NSC682769 treated animals (5 mg/kg; 20 mg/kg) as compared to vehicle treated animals consistent with the observed effects of the inhibitor in vitro (FIG. 5D; see also FIG. 3). Expression of the YAP-dependent genes CTGF and Cyr61 was reduced in inhibitor treated groups relative to vehicle (FIG. 5E). A reduction in Ki-67 staining of harvested tumors from animals treated with NSC682769 was evident as compared to vehicle treated mice (FIG. 5F) and enhancement of apoptotic death was observed via TUNEL staining at both dosing regimens, supporting the increased rate of apoptosis observed in vitro (FIG. 5G, see also FIG. 3E).

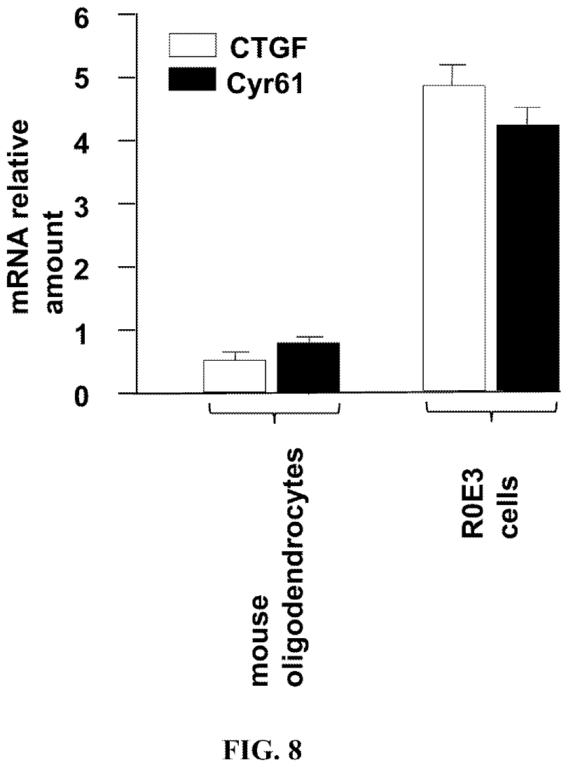

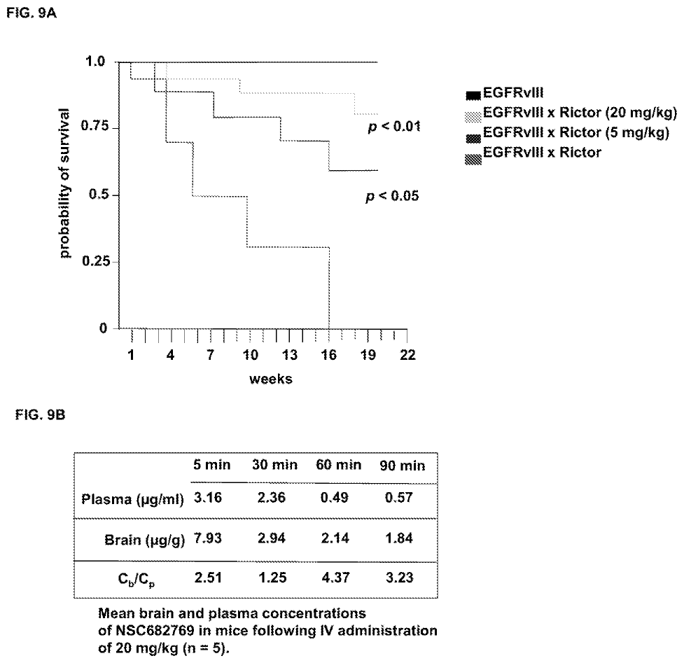

NSC682769 increases survival of a glioma GEMM and penetrates the blood-brain barrier. NSC682769 was evaluated in double transgenic GFAP-EGFRvIII; GFAP-Cre/Rictor^{loxP/loxP} mice which overexpress both the mutant constitutively active EGFRvIII allele and the mTORC2 scaffolding component Rictor. These mice develop high-grade bilateral, multifocal, infiltrating mixed astrocytic-oligodendroglial tumors displaying elevated mTORC2 signaling with nearly complete penetrance (Bashir T, et al. (2012) PLoS One 7: e47741). In a cell line derived from oligodendroglial tumors from these mice (R0E3 cells) YAP-dependent gene expression was elevated (FIG. 8). To assess therapeutic activity, the effect of twice-weekly 5 or 20 mg/kg NSC682769 treatments was measured on survival. As shown in FIG. 9A, approximately 50% of GFAP-EGFRvIII×GFAP-Cre⁺/Rictor mice developed gliomas by 5-7 weeks and the mice succumbing by 16 weeks. Importantly, NSC682769 treated GFAP-EGFRvIII×GFAP-Cre⁺/Rictor mice had a marked increase in overall survival with more than 75% of mice surviving at 20 weeks at 20 mg/kg and 60% of mice surviving to 20 weeks receiving the lower 5 mg/kg regimen.

While GBMs may partially disrupt the BBB by inducing large gaps between endothelial cells, the extent of disruption among individual patients and/or among various regions within a single tumor are highly variable. Thus, it was determined whether NSC682769 would cross the BBB by developing an HPLC method to quantify the inhibitor in brain tissue and serum. In C57/BL6 mice, following a single IV administration of NSC682769, the compound accumulated to significant levels with a peak value of 6.22 μg/g at 5 min post-administration (FIG. 9B). C_{b}/Cₚ ratios for NSC682769 are consistent with BBB penetrance.

Discussion. The Hippo signaling pathway has received considerable attention recently and has been implicated as an attractive therapeutic target in GBM (Orr B A, et al. (2011) J Neuropathol Exp Neurol 70: 568-577; Shao D D, et al. (2014) Cell 158: 171-184; Thompson B J (2020) Bioessays 42: e1900162; and Artinian N, et al. (2015) J Biol Chem 290: 19387-19401). Evidence suggests that deregulation of Hippo signaling and activation of its downstream effector YAP leads to increased GBM cell growth and motility (Orr B A, et al. (2011) J Neuropathol Exp Neurol 70: 568-577; and Artinian N, et al. (2015) J Biol Chem 290: 19387-19401). As described herein, a potent small molecule inhibitor was identified and it demonstrated marked inhibitory effects on YAP/TEAD transcriptional activity and anti-GBM properties on cell lines and patient-derived cells. NSC682769 appears to directly bind to YAP and inhibits its association with the four TEAD family members. Moreover, sensitivity to NSC682769 directly correlated with an elevated degree of nuclear YAP expression. NSC682769 also enhanced the survival of a transgenic glioma mouse model in a dose-dependent manner and determinations of mean brain and blood plasma concentrations following single IV administration of the inhibitor suggested clear blood-brain barrier penetrance.

These data are consistent with a working model in which NSC682769 attenuates YAP expression by binding to YAP and resulting in targeted degradation of the protein. This is indirectly supported by both the cell fractionation and immunolocalization studies. In LN229 and GBM39 cells, treatment with NSC682769 resulted in a marked cytoplasmic redistribution of nuclear YAP (see, FIGS. 2F&G) where normally, the S127 phosphorylated protein binds to 14-3-3 proteins resulting in their cytoplasmic retention and suppressed target gene transcription. Further serine phosphorylation of YAP by casein kinase 1, leads to subsequent proteasomal degradation (Zhao B, et al. (2010) Genes Dev 24: 72-85). It is possible that NSC682769-bound YAP adopts a conformation enhancing 14-3-3 binding, leading to proteasomal-mediated turnover.

It has been suggested that the development of orthosteric small molecule inhibitors of the YAP-TEAD protein-protein interface may be difficult owing to the interaction occurring over a large and mostly featureless interaction interface (˜1,300 Å²) with a K_{d} in the nanomolar range (Bum-Erdene K, et al. (2019) Cell Chem Biol 26: 378-389). While this may be the case, it is possible that small structural pockets may exist which are competent to bind small probe compounds. The NSC682769 affinity bead pull-down and SPR analyses demonstrate that the compound binds to YAP (see FIGS. 1F&G). In this regard, verteporfin, identified as a prototypic YAP-TEAD interaction inhibitor (Liu-Chittenden Y, et al. (2012) Genes Dev 26: 1300-1305), has not been demonstrated to bind either YAP or TEAD and its inhibitory effects on YAP signaling may be due to non-specific effects (Liu-Chittenden Y, et al. (2012) Genes Dev 26: 1300-1305). While verteporfin did inhibit YAP-TEAD association in the in vitro experiments, the blockade required markedly higher levels of verteporfin to interfere with the interaction as compared to NSC682769 exposure (see, FIG. 1E). Future binding site mapping, in silico docking and mutagenesis experiments will shed light on the detailed mechanism of action of NSC682769.

In conclusion, these studies demonstrate that NSC682769 represents a newly identified inhibitor of the YAP-TEAD protein-protein interaction targeting GBMs with elevated YAP expression. NSC682769 appears to have significant inhibitory effects on YAP-TEAD dependent transcription and markedly inhibits GBM cell growth.

What is claimed is:

1. A method of treating cancer in a subject, the method comprising:
administering to the subject a therapeutically effective amount of compound capable of inhibiting the binding between Yes-associated protein (YAP) and TEA domain (TEAD), thereby reducing YAP levels.

2. A method of reducing Yes-associated protein (YAP) levels in a subject, the method comprising: administering to the subject a therapeutically effective amount of compound capable of inhibiting the binding between Yes-associated protein (YAP) and TEA domain (TEAD), thereby reducing YAP levels.

3. A method of reducing Yes-associated protein (YAP) expression in a subject, the method comprising: administering to the subject a therapeutically effective amount of compound capable of inhibiting the binding between Yes-associated protein (YAP) and TEA domain (TEAD), thereby reducing YAP expression.

4. A method of inhibiting cell proliferation, cell migration, or cell invasiveness, the method comprising: contacting a cell or tissue or administering to a subject a therapeutically effective amount of compound capable of inhibiting the binding between Yes-associated protein (YAP) and TEA domain (TEAD), thereby reducing YAP levels.

5. A method of inhibiting tumor growth, the method comprising: contacting a cell or tissue or administering to the subject a therapeutically effective amount of compound capable of inhibiting the binding between Yes-associated protein (YAP) and TEA domain (TEAD), thereby reducing YAP levels.

6. A method of treating glioblastoma in a subject, the method comprising: administering to the subject a therapeutically effective amount of compound capable of inhibiting the binding between Yes-associated protein (YAP) and TEA domain (TEAD), thereby reducing YAP levels.

7. The method of any of the preceding claims, further comprising administering a therapeutically effective amount of a chemotherapeutic agent.

8. The method of any of the preceding claims, wherein the subject is identified as being in need of treatment before the administration step.

9. The method of any of the preceding claims, wherein the subject has been identified as having elevated YAP levels.

10. The method of any of the preceding claims, wherein the compound is

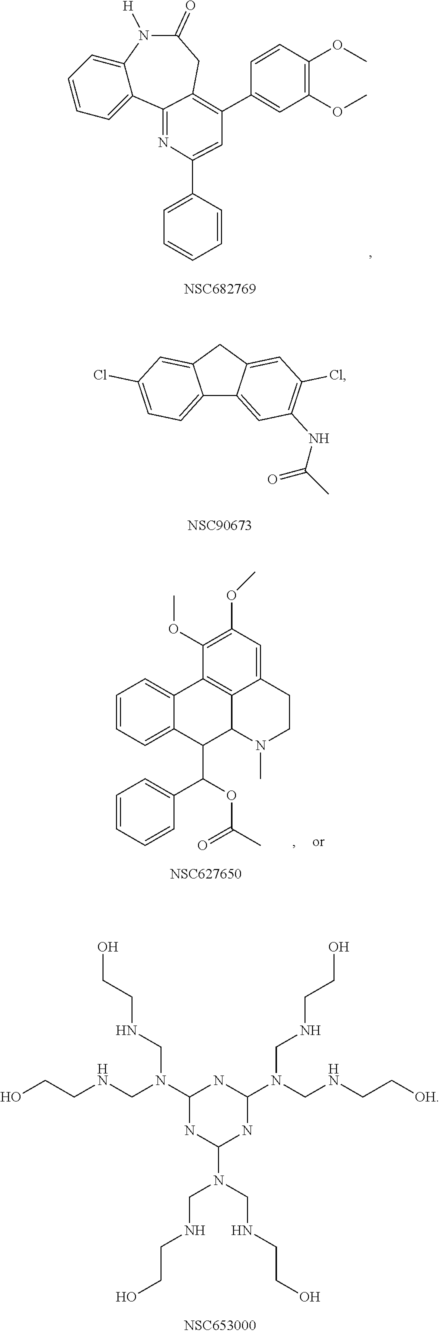 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]

11. The method of any of the preceding claims, the subject has been diagnosed with cancer prior to the administering step.

12. The method of claim 1, wherein the cancer is a primary or secondary tumor.

13. The method of claim 12, wherein the primary or secondary tumor is within the subject's breast, brain, lung, liver, pancreas, prostate or thyroid.

14. The method of claim 1, wherein the cancer is brain cancer, breast cancer, lung cancer, liver cancer, pancreatic cancer, prostate cancer or thyroid cancer.

15. The method of claim 14, wherein the brain cancer is glioblastoma.

16. The method of any of the preceding claims, wherein the subject is a human.

17. The method of any of the preceding claims, wherein the therapeutically effective amount of the compound is administered orally or parentally.

18. The method of claim 17, wherein the parenteral administration is intravenous, subcutaneous, intramuscular, intrathecal or direct injection.

19. A method comprising:
a) obtaining or having obtained a sample comprising tumor cells from a cancer patient;
b) measuring the level of Yes-associated protein (YAP) in the tumor cells of the sample;
c) identifying the cancer patient as a suitable candidate for treatment with a composition comprising: 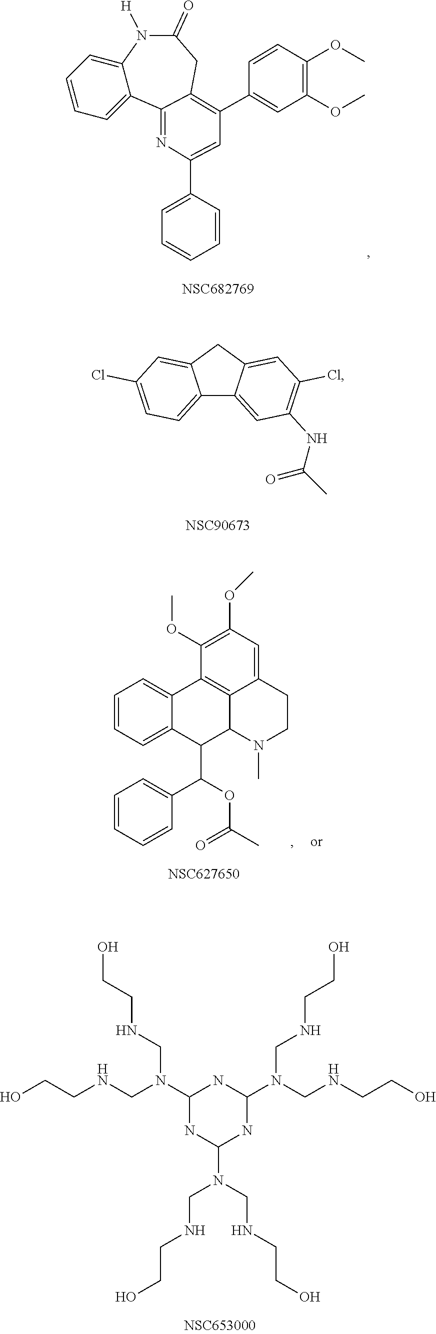 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]
when the level of YAP is higher than a level of YAP in a control sample and identifying the cancer patient as an unsuitable candidate for treatment with a composition comprising 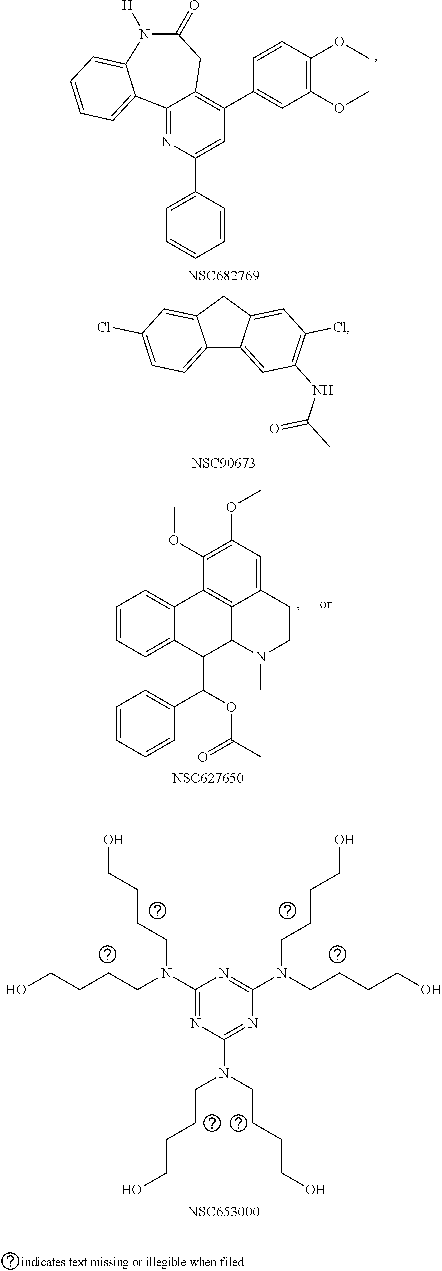 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCCCN(CCCCO)c1nc(N(CCCCO)CCCCO)nc(N(CCCCO)CCCCO)n1]
when the level of YAP is the same or lower than a level in a control sample; and
d) administering the composition comprising 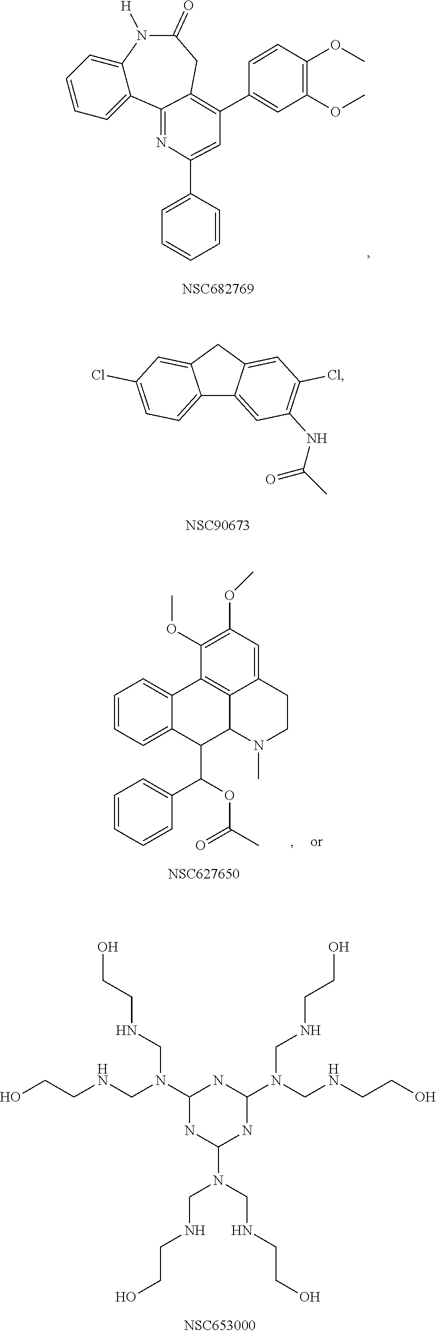 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]
to the cancer patient identified as the suitable candidate, and not administering the composition comprising 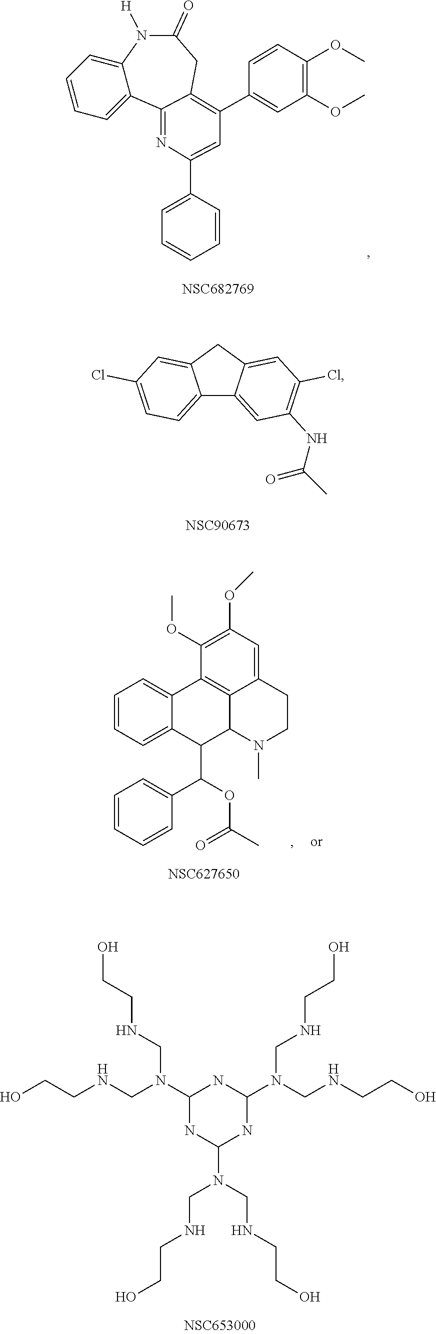 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]
to the cancer patient identified as the unsuitable candidate.

20. The method of claim 19, wherein the patient has brain cancer, breast cancer, lung cancer, liver cancer, pancreatic cancer, prostate cancer or thyroid cancer.

21. The method of claim 20, wherein the brain cancer is glioblastoma.

22. The method of any of claims 19-21, wherein the sample is a biopsy.

23. The method of any of claims 19-22, wherein the composition comprising 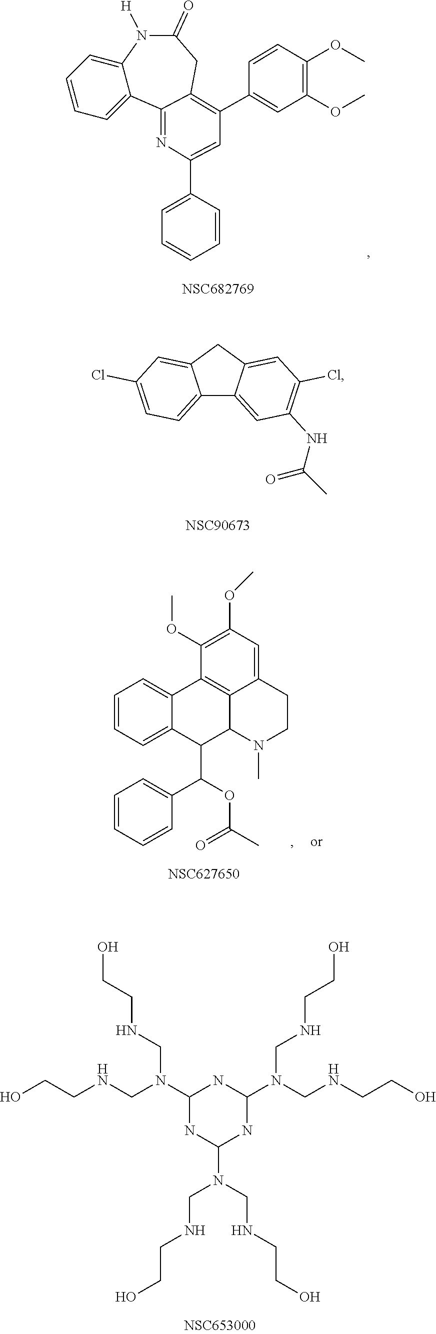 [SMILES: COc1ccc(-c2cc(-c3ccccc3)nc3c2CC(=O)Nc2ccccc2-3)cc1OC ; CC(=O)Nc1cc2c(cc1Cl)Cc1cc(Cl)ccc1-2 ; COc1cc2c3c(c1OC)-c1ccccc1C(C(OC(C)=O)c1ccccc1)C3N(C)CC2 ; OCCNCN(CNCCO)C1NC(N(CNCCO)CNCCO)NC(N(CNCCO)CNCCO)N1]
is administered systemically.

24. The method of any of claims 19-23, further comprising administering a therapeutically effective amount of a chemotherapeutic agent.
